(19) United States
(12) Patent Application Publication
(10) Pub. No.: US 2008/0255076 A1
(43) Pub. Date: Oct. 16, 2008

(54) Steroid-Derived Pharmaceutical Compositions

(75) Inventors: Tobias Braxmeier, Dresden (DE); Kai Simons, Dresden (DE); Marino Zerial, Dresden (DE); Teymuras Kurzchalia, Dresden (DE); Tim Friedrichson, Dresden (DE); Wolfgang Frohner, Walldorf (DE); Gary Jennings, Dresden (DE); Georg Schlechtingen, Dresden (DE); Cornelia Schroeder, Berlin (DE); Hans-Joachim Knolker, Dresden (DE)

Correspondence Address: FULBRIGHT & JAWORSKI L.L.P., 600 CONGRESS AVE., SUITE 2400, AUSTIN, TX, 78701, US

(73) Assignees: JadoLabs GmbH, Dresden (DE); Max-Planck-Gesellschaft zur Forderung der Wissenschaftenhaften e.V., Berlin (DE); Technische Universitat Dresden, Dresden (DE)

(21) Appl. No.: 11/571,345

(22) PCT Filed: Jun. 29, 2005
(86) PCT No.: PCT/EP2005/007031

§ 371 Date: Jan. 24, 2008

Related U.S. Application Data
(60) Provisional application No. 60/636,840, filed on Dec. 16, 2004.

(30) Foreign Application Priority Data
Jun. 29, 2004 (EP) 04015249.8

(51) Int. Cl. A61K 31/57; A61K 31/575; A61K 31/565; A61P 35/00; A61P 25/28; A61P 31/12; A61P 31/04; A61P 33/00

(52) U.S. Cl. 514/177; 514/169; 514/182

(57) ABSTRACT

The present invention relates to the use of specific steroid derivatives in the preparation of medicaments for the treatment or prevention and/or amelioration of disorders relating to pathological processes in lipid rafts.

The present invention relates to the use of specific steroid derivatives in the preparation of medicaments for the treatment or prevention and/or amelioration of disorders relating to pathological processes in lipid rafts.

The lipid bilayer that forms cell membranes is a two dimensional liquid the organization of which has been the object of intensive investigations for decades by biochemists and biophysicists. Although the bulk of the bilayer has been considered to be a homogeneous fluid, there have been repeated attempts to introduce lateral heterogeneities, lipid microdomains, into our model for the structure and dynamics of the bilayer liquid (Glaser, Curr. Opin. Struct. Biol. 3 (1993), 475-481; Jacobson, Comments Mol. Cell Biophys. 8 (1992), 1-144; Jain, Adv. Lipid Res. 15 (1977), 1-60; Winchil, Curr. Opin. Struct. Biol. 3 (1993), 482-488.

The realization that epithelial cells polarize their cell surfaces into apical and basolateral domains with different protein and lipid compositions in each of these domains, initiated a new development that led to the “lipid raft” concept (Simons, Biochemistry 27 (1988), 6197-6202; Simons, Nature 387 (1997), 569-572). The concept of assemblies of sphingolipids and cholesterol functioning as platforms for membrane proteins was promoted by the observation that these assemblies survived detergent extraction, and are referred to as detergent resistant membranes, DRM (Brown, Cell 68 (1992), 533-544). This was an operational break-through where raft-association was equated with resistance to Triton-X100 extraction at 4° C. The addition of a second criterion, depletion of cholesterol using methyl-β-cyclodextrin (Ilangumaran, Biochem. J. 335 (1998), 433-440; Scheiffele, EMBO J. 16 (1997), 5501-5508), leading to loss of detergent resistance, prompted several groups in the field to explore the role of lipid microdomains in a wide spectrum of biological reactions. There is now increasing support for a role of lipid assemblies in regulating numerous cellular processes including cell polarity, protein trafficking and signal transduction.

Cell membranes are two-dimensional liquids. Thus, lateral heterogeneity implies liquid-liquid immiscibility in the membrane plane. It has been well known that hydrated lipid bilayers undergo phase transitions as a function of temperature. These transitions, which occur at defined temperatures for each lipid species, always involve some change in the order of the system. The most important of these transitions is the so-called “main” or “chain-melting” transition in which the bilayer is transformed from a highly ordered quasi-two dimensional crystalline solid to a quasi-two dimensional liquid. It involves a drastic change in the order of the systems, in particular of the translational (positional) order in the bilayer plane and of the conformational order of the lipid chains in a direction perpendicular to this plane. Translational order is related to the lateral diffusion coefficient in the plane of the membrane and conformational order is related to the trans/gauche ratio in the acyl chains. The main transition has been described as an ordered-to-disordered phase transition, so that the two phases may be labeled as solid-ordered (sₒ) below the transition temperature and liquid-disordered (l_{d}) above that temperature. Cholesterol and phopholipids are capable of forming a liquid-ordered (lₒ)) phase that can coexist with a cholesterol-poor liquid-disordered (l_{d}) phase thereby permitting phase coexistence in wholly liquid phase membranes (Ipsen, Biochem. Biophys. Acta 905 (1987) 162-172; Ipsen, Biophys. J. 56 (1989), 661-667). Sterols do so as a result of their flat and rigid molecular structure, which is able to impose a conformational ordering upon a neighboring aliphatic chain (Sankaram, Biochemistry 29 (1990), 10676-10684), when the sterol is the nearest neighbor of the chain, without imposing a corresponding drastic reduction of the translational mobility of the lipid (Nielsen, Phys. Rev. E. Stat. Phys. Plasmas Fluids Relat. Interdiscip. Topics 59 (1999), 5790-5803). Due to the fact that the sterol does not fit exactly in the crystalline lattice of an sₒ (gel) lipid bilayer phase it will, if it dissolves within this phase, disrupt the crystalline translational order without, however, significantly perturbing the conformational order. Thus, cholesterol at adequate molar fractions can convert l_{d} or sₒ lipid bilayer phases to liquid-ordered (lₒ) phases.

Lipid rafts are lipid platforms of a special chemical composition (rich in sphingomyelin and cholesterol in the outer leaflet of the cell membrane) that function to segregate membrane components within the cell membrane. Rafts are understood to be relatively small (30-50 nm in diameter, estimates of size varying considerably depending on the probes used and cell types analysed) but they can be coalesced under certain conditions. Their specificity with regard to lipid composition is reminiscent of phase separation behavior in heterogeneous model membrane systems. In fact, many of their properties with regard to chemical composition and detergent solubility are similar to what is observed in model systems composed of ternary mixtures of an unsaturated phosphatidylcholine, sphingomyelin (or a long-chain saturated phosphatidylcholine), and cholesterol (de Almeida, Biophys. J. 85 (2003), 2406-2416). Rafts may be considered domains of a lₒ phase in a heterogeneous l phase lipid bilayer composing the plasma membrane. What the other coexisting phase (or phases) is (or are) is not clear at present. There is consensus that the biological membrane is a liquid, so sₒ phase coexistence may be ignored for most cases. Whether the other phase (phases) is (are) l_{d} or lₒ phases will depend upon the chemical identity of the phospholipids that constitute this phase (these phases) and the molar fraction of cholesterol in them. Rafts may be equated with a liquid-ordered phase and refer to the rest of the membrane as the non-raft liquid phase. Within the framework of thermodynamics, a phase is always a macroscopic system consisting of large number of molecules. However, in lipid bilayers the phases often tend to be fragmented into small domains (often only a few thousand molecules) each of which, per se, may not have a sufficient number of molecules to strictly satisfy the thermodynamic definition of a phase. The liquid-ordered raft phase thus comprises all the domains (small or clustered) of the raft phase in the membranes. The rest of the membrane surrounding the rafts, the liquid phase, may be a homogeneous percolating liquid phase or may be further subdivided into liquid domains not yet characterized.

Pralle, J. Cell. Biol. (2000) 148, 997-1008 employed photonic force microscopy to measure the size of lipid rafts and found that rafts in the plasma membrane of fibroblasts diffuse as assemblies of 50 nm diameter, corresponding to a surface area covered by about 3,000 sphingolipids. Based on data from cultured baby hamster kidney (BHK) cells, whose lipid composition and organelle surface area have been examined in detail, it appears that an individual cell has a surface area of approximately 2,000 μm². The lipid composition of the cell plasma membrane contains 26% phosphatidylcholine, 24% sphingomyelin, and 12% glycosphingolipids. Due to the asymmetric nature of the lipid organization in the plasma membrane, most of the sphingolipids occupy the outer leaflet of the bilayer, while less than half of the phosphatidylcholine has been estimated to be in this leaflet.

Assuming that most of the sphingolipid is raft-associated, rafts would cover more than half of the cell surface. The density of membrane proteins has been estimated to be around 20,000 molecules per μm². Thus, the plasma membrane would accordingly contain about 40×10⁶ protein molecules. The number of 50-nm rafts would be about 10⁶, and if the density of proteins is the same in rafts as in the surrounding bilayer, each raft would carry about 20 protein molecules. If BHK cells are representative, it follows that the density of rafts floating in the fibroblast plasma membrane is high. If 20×10⁶ raft protein molecules were distributed more or less randomly, each raft would likely contain a different subset of proteins. A kinase attached to the cytosolic leaflet of a raft is, therefore, unlikely to meet its substrate in the same individual raft. The small size of an individual raft may be important for keeping raft-borne signaling proteins in the “off” state. Accordingly, for activation to occur, many rafts have to cluster together, forming a larger platform, where the protein participants in a signal transduction process can meet, undisturbed by what happens outside the platform. Thus, rafts are small, and, when activated, they cluster to form larger platforms in which functionally related proteins can interact. One way to analyze raft association and clustering is to patch raft and nonraft components on the surface of living cells by specific antibodies (Harder, J Cell Biol. 141 (1998), 929-942; Janes, Semin. Immunol. 12 (2000), 23-34). If two raft components are cross-linked by antibodies,they will form overlapping patches in the plasma membrane. However, patching of a raft protein and a nonraft marker such as the transferrin receptor leads to the formation of segregated patches. Co-patching of two raft components is dependent on the simultaneous addition of both antibodies to the cells. If antibodies are added sequentially, segregated patches predominate. Notably, the patching behavior is cholesterol-dependent. As a consequence of the small size and the heterogeneous composition of individual rafts, these structures must be clustered in specific ways if signaling is to ensue. One example of such a raft clustering process encountered in daily clinical practice is the IgE signaling during the allergic immune response (Sheets, Curr. Opin. Chem. Biol. 3 (1999), 95-99; Holowka, Semin. Immunol. 13 (2001), 99-105). The allergen that elicits the allergic reaction by stimulating the degranulation of a mast or basophilic cell is multivalent, binding several IgE antibody molecules. Cross-linking of two or more IgE receptors [Fc(ε)RI] increases their association with rafts, as measured by increased detergent resistance. Within the rafts, cross-linked Fc(ε)RI becomes tyrosine phosphorylated by raft-associated Lyn, a double-acylated Src-related kinase. The Fc(ε)RI phosphorylation recruits Syk-related kinases, which are activated and lead to binding and scaffolding of downstream signaling molecules and, finally, to the formation of a signaling platform. This structure includes the raft protein LAT (linker of activation of T cells), which guides the clustering of additional rafts into the expanding platform (Rivera, Int. Arch. Allergy Immunol. 124 (2001), 137-141). Signaling leads to calcium mobilization, which triggers the release of preformed mediators such as histamine from the intracellular stores. The more participants are collected into the raft platform, the higher the signaling response. Uncontrolled amplification of the signaling cascade by raft clustering might trigger hyperactivation, with life-threatening consequences such as Quinke edema and allergic shock. The whole signaling assembly can be dissociated by dephosphorylation or downregulated by internalization of the components by endocytosis (Xu, J. Cell Sci. 111 (1998), 2385-2396). Thus, in IgE signaling, lipid rafts serve to increase the efficiency by concentrating the participating proteins into fluid microdomains and limiting their lateral diffusion so that proteins remain at the site of signaling. Even a small change of partitioning into lipid rafts can, through amplification, initiate a signaling cascade or prompt a deleterious overshoot, as occurs in allergic reactions (Kholodenko, Trends Cell Biol. 10 (2000), 173-178). Another clinically relevant example of raft clustering is the pathogenic mechanisms of pore-forming toxins, which are secreted by Clostridium, Streptococcus, and Aeromonas species, among other bacteria. These toxins may cause diseases ranging from mild cellulites to gaseous gangrene and pseudomembranous colitis. Best studied is the toxin aerolysin from the marine bacterium Aeromonas hydrophila. Aerolysin is secreted and binds to a GPI-anchored raft protein on the surface of the host cell. The toxin is incorporated into the membrane after proteolysis and then heptamerizes in a raft-dependent manner to form a raft-associated channel through which small molecules and ions flow to trigger the pathogenic changes. The oligomerization of aerolysin can be triggered in solution but occurs at more than 10⁵-fold lower toxin concentration at the surface of the living cell. This enormous increase in efficiency is due to activation by raft binding and by concentration into raft clusters, which is driven by the oligomerization of the toxin. Again, a small change can lead to a huge effect by amplification of raft clustering (Lesieur, Mol. Membr. Biol. 14 (1997), 45-64; Abrami, J. Cell Biol. 147 (1999), 175-184).

Lipid rafts contain specific sets of proteins (van Meer, Annu. Rev. Cell Biol. 5 (1989), 247-275; Simons, Annu. Rev. Biophys. Biomol. Struct. 33 (2004), 269-295). These include, inter alia, GPI-anchored proteins, doubly acylated proteins such as tyrosine kinases of the src family, Gα subunits of heteromeric G proteins and endothelial nitric oxide synthase, the cholesterol- and palmitate-linked hedgehog protein and other palmitate-linked proteins, as well as transmembrane proteins. Proteins with attached saturated acyl chains and cholesterol can be associated with liquid-ordered raft domains. Studies with model membranes have confirmed that peptides containing such lipid modifications associate with liquid-ordered domains (Wang, Biophys. J. 79 (2000,) 919-933). It should be noted that the GPI anchors differ in their fatty acid composition. Some GPI anchors contain unsaturated acyl chains, and how these interact with lipid rafts remains to be studied.

Transmembrane proteins, since they cross the bilayer, may disrupt the packing of the liquid-ordered domain. Yet, the lₒ phase is a liquid phase and therefore does not have long-range order in the membrane plane. Association of proteins with lipid rafts can be viewed as a simple solubility problem described by an equilibrium partition coefficient for partitioning of the protein between two coexisting phases, or it can be understood to require some chemical affinity for raft lipids. Several proteins interact with cholesterol. Caveolin is the prime example (Murata, Proc. Natl. Acad. Sci. USA 92 (1995), 10339-10343). There are also examples of receptor proteins interacting with glycosphingolipids including gangliosides (Hakomori, Proc. Natl. Acad. Sci. USA 99 (2002), 225-232). A structural protein motif has been identified for binding to sphingolipids (Mahfoud, J. Biol. Chem. 277 (2002), 11292-11296). Recent results also demonstrate that proteins can exist in different states depending on the membrane environment. Glutamate receptors, which are G protein-coupled heptahelical transmembrane proteins, are in a low-affinity state when reconstituted into membranes lacking cholesterol. The receptor changes its conformation in liquid-ordered cholesterol-containing membranes and now binds its ligand with high affinity (Eroglu, Proc. Natl. Acad. Sci. USA. 100 (2003), 10219-10124). The EGF receptor is activated by interaction with the ganglioside GM3 and inactivated by cholesterol depletion (Miljan, Sci. STKE. 160 (2002), 15). The receptor seems to depend on the lipid environment for high-affinity binding capability. One way to view this differential behavior would be to consider the protein as a solute in the bilayer solvent of the membrane. If the lipid bilayer has two phases, each phase is a different solvent. The protein has a conformation that depends on its environment and therefore depends on the bilayer solvent phase in which it is dissolved. So one can expect that in a nonraft domain it will have one conformation, and in the raft domain it will have another. The receptor activation would depend on the partition coefficient between the different lipid domains in the bilayers and upon phase coexistence. Another issue is the length of the transmembrane domains of the protein, because a liquid-ordered bilayer is thicker than a liquid-disordered one. These parameters play a role in protein sorting to the cell surface (Bretscher, Science 261 (1993), 1280-1281). But how precisely the transmembrane domains should be matched with the thickness of the bilayer is an open issue. So far, no detailed analysis has been carried out of how different transmembrane proteins having different transmembrane domain lengths partition into liquid-ordered and liquid-disordered domains. The transmembrane domains of single-span transmembrane proteins in the plasma membrane are usually longer than the transmembrane domains of proteins that reside in the Golgi complex or in the endoplasmic reticulum.

Anderson, Mol. Biol. Cell 7 (1996), 1825-1834 demonstrates that treatment of CV-1 or HeLa cells with the phorbol ester PMA or the macrolide polyene antibiotics Nystatin and Filipin blocked infection by Simian Virus 40 (SV40) in a reversible manner. Phorbol esters, well-known tumor promoters, are activators of protein kinase C and disrupt caveolae by blocking their invaginations (Smart (1994) J. Cell Biol. 124, 307-313). The cholesterol-binding drugs Nystatin and Filipin represent members of the polyene antimycotica, such as the structurally similar Amphotericin B, and are widely used in standard therapy for the treatment of fungal infections. Anderson and colleagues speculate that the selective disruption of caveolae due to cholesterol depletion by those drugs is causal for the observed effect and that caveolae might mediate virus entry.

Gidwani, J. Cell Sci. 116 (2003), 3177-3187 describes an in vitro assay employing specific amphiphiles to disrupt lipid rafts. It is speculated that certain ceramides may serve as useful probes for investigating the role of plasma membrane structure and of phospholipase D activity in cellular signaling.

Wang, Biochemistry 43 (2004), 1010-1018 investigates the relationship between sterol/steroid structures and participation in lipid rafts. These authors consider this question of interest, since sterols may be used to distinguish biological processes dependent on cholesterol in cells from those processes that can be supported by any raft environment. Interestingly, Wang and colleagues have found steroids which promoted the formation of ordered domains in biological membranes.

WO 01/22957 teaches the use of gangliosides for the modulation of sphingolipid/cholesterol microdomains and it is taught that gangliosides provoke a modulation of rafts by displacement/replacement of proteins, in particular GPI-APs. It is speculated that gangliosides, ganglioside derivatives or cholesterol derivatives may be used in a clinical setting to modulate the sphingolipid-cholesterol microdomain in particular by influencing the location of anchor proteins, acetylated proteins, kinases and/or cholesterol anchor proteins.

A problem underlying the present invention is the provision of means and methods for clinical and/or pharmaceutical intervention in disorders linked to and/or associated with biological/biochemical processes regulated by lipid rafts.

The solution to this technical problem is achieved by providing the embodiments characterized herein below as well as in the claims.

Accordingly, the present invention provides for the use of a compound of one of the following formulae 1a, 1b, 1c and 1d;

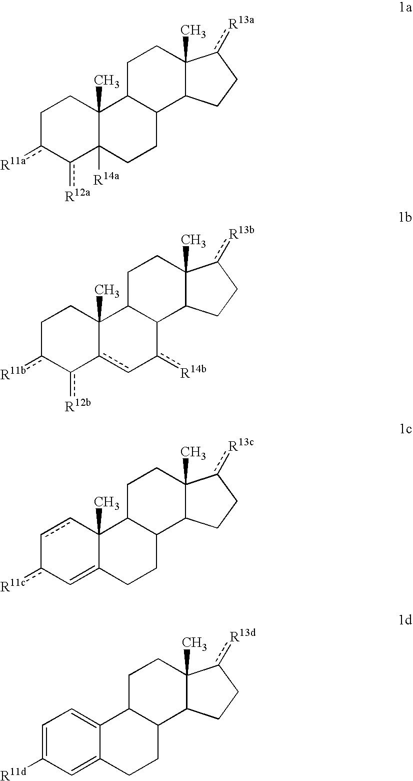

or a pharmaceutically acceptable salt, derivative, solvate or prodrug thereof for the preparation of a pharmaceutical composition for the treatment, prevention and/or amelioration of a disease/disorder caused by a biochemical/biophysical pathological process occurring on, in or within lipid rafts.

The following numbering of the carbon atoms and denotation of the rings of the steroid scaffold will be adhered to throughout the description:

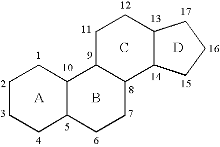

In the formulae provided herein, [symbol] is used to represent a single bond or a double bond, and [symbol] is employed to denote a single bond, a double bond or a triple bond.

Furthermore, the general formulae given in the present invention are intented to cover all possible stereoisomers and diastereomers of the indicated compounds. Unless indicated differently, the stereochemical configuration of naturally occurring cholesterol is preferred.

R^{11a}, R^{11b} and R^{11c} are H, OR, NR₂, N₃, SO₄⁻, SO₃⁻, PO₄²⁻, halogen, O or S, provided that if R^{11a}, R^{11b} or R^{11c} is O or S then the bond connecting said R^{11a}, R^{11b} or R^{11c} to the ring system is a double bond, in all other cases said bond is a single bond. Preferably, R^{11a}, R^{11b} and R^{11c} are OH, O(C₁₋₄ alkyl), NR₂, SO₄⁻, SO₃⁻or O. More preferably, R^{11a}, R^{11b} and R^{11c} are OH, OCH₃, NH₂, N(C₁₋₄ alkyl)₂, SO₄⁻or O.

R^{11d} is OR, NR₂, SO₄⁻, PO₄²⁻, COOH, CONR₂ or OCO(C₁₋₄ alkyl). Preferably, R^{11d} is OR, NR₂, COOH or OCO(C₁₋₂ alkyl). More preferably, R^{11d} is OCH₃, NR₂ or OCOCH₃.

Each R is independently H or C₁₋₄ alkyl.

R^{12a}and R^{12b} are H, OR, NR₂, N₃, halogen or O, provided that if R^{12a} or R^{12b} is O then the bond connecting said R^{12a} or R^{12b} to the ring system is a double bond, in all other cases said bond is a single bond. Preferably, R^{12a} and R^{12b} are H, O(C₁₋₄ alkyl), halogen or O.

R^{11a} and R^{12a} are not simultaneously H and R^{11b} and R^{12b} are not simultaneously H. If both R^{11a} and R^{12a} are bonded to the ring system via a single bond and both are not H, they are preferably in an anti orientation to each other. If both R^{11b} and R^{12b} are bonded to the ring system via a single bond and both are not H, they are preferably in an anti orientation to each other.

R^{13a}, R^{13b}, R^{13c} and R^{13d} are H; C₁₋₅ alkyl, wherein one or more hydrogens are optionally replaced by halogen; C₁₂₋₂₄ alkyl, wherein one or more hydrogens are optionally replaced by halogen, preferably C₁₂₋₁₈ alkyl, wherein one or more hydrogens are optionally replaced by halogen; C₁₋₅ alkylidene, wherein one or more hydrogens are optionally replaced by halogen; C₁₂₋₂₄ alkylidene, wherein one or more hydrogens are optionally replaced by halogen, preferably C₁₂₋₁₈ alkylidene, wherein one or more hydrogens are optionally replaced by halogen; C₂₋₅ alkenyl, wherein one or more hydrogens are optionally replaced by halogen; C₂₋₅ alkynyl, wherein one or more hydrogens are optionally replaced by halogen; 1-adamantyl; (1-adamantyl)methylene; C₃₋₈ cycloalkyl, wherein one or more hydrogens are optionally replaced by halogen; (C₃₋₈ cycloalkyl)methylene, wherein one or more hydrogens are optionally replaced by halogen; provided that if R^{13a}, R^{13b} or R^{13c} is C₁₋₅ alkylidene or C₁₂₋₂₄ alkylidene then the bond connecting said R^{13a}, R^{13b} or R^{13c} to the ring system is a double bond, in all other above-mentioned cases said bond is a single bond.

Alternatively, R^{13a}, R^{13b} and R^{13c} are a group of the following formula 2:

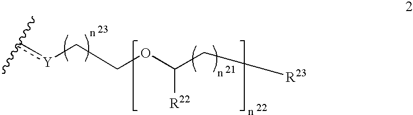

R²³ is O—R²¹. R²³ is also envisaged to be NH—R²⁴.

R²¹ is C₁₋₄ alkyl, preferably CH₃. R²¹ is also envisaged to be CO(C₁₋₄ alkyl) or H. Preferably, R²¹ is CH₃ or COCH₃.

R²⁴ is C₁₋₄ alkyl, CO(C₁₋₄ alkyl) or H. Preferably, R²⁴ is CH₃, COCH₃ or H.

Each R²² is independently H or C₁₋₃ alkyl, preferably H or CH₃.

Y is CH₂, CH or O, provided that if Y is CH then the bond connecting Y to the ring system is a double bond, in all other cases said bond is a single bond. Preferably, Y is CH₂ or O.

Each n²¹ is independently an integer of 1 or 2, preferably 1.

n22 is an integer from 0 to 5, preferably from 1 to 4.

If Y is O then n²³ is 1, in all other cases n²³ is 0.

Preferably, R^{13a} R^{13b} R^{13c} and R^{13d} are H, C₁₋₅ alkyl, C₁₋₅ alkylidene, C₁₂₋₁₄ alkyl or C₁₂₋₁₄ alkylidene. In another preferred embodiment, R^{13a}, R^{13b}, R^{13c} and R^{13d} are the group of formula 2.

R^{14a} , is H. In one embodiment, R^{14a} is in the beta-orientation, i.e. R^{14a} and the CH₃ group in the 10 position of the steroid scaffold of compound 1a are cis to each other. However, compounds wherein R^{14a} is in the alpha-orientation, i.e. R^{14a} and the CH₃ group in the 10 position of the steroid scaffold of compound 1a are trans to each other are also envisaged.

R^{14b} is H, OR, halogen or O, provided that if R^{14b} is O then the bond connecting R^{14b} to the ring system is a double bond, in all other cases said bond is a single bond. Preferably, R^{14b} is H, halogen or O.

Also provided in accordance with the invention is the use of a compound of the following formula 3:

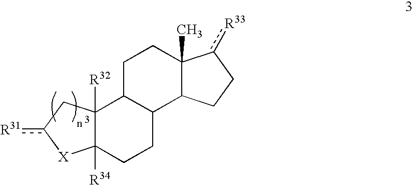

or a pharmaceutically acceptable salt, solvate or prodrug thereof for the preparation of a pharmaceutical composition for the treatment, prevention and/or amelioration of a disease/disorder caused by a biochemical/biophysical pathological process occurring on, in or within lipid rafts.

R³¹ is H, halogen or O, provided that if R³¹ is O then the bond connecting R³¹ to the ring system is a double bond, in all other cases said bond is a single bond.

In one embodiment, X is O. In another embodiment, X is N—R³⁵. If X is O, then R³¹ is preferably H. If X is N—R³⁵, then R³¹ is preferably H or O, more preferably O.

R³⁵ is H or C₁₋₄ alkyl, preferably C₁₋₄ alkyl, more preferably CH₃.

R³² is H or CH₃, preferably CH₃.

R³³ is H; C₁₋₅ alkyl, wherein one or more hydrogens are optionally replaced by halogen; C₁₂₋₂₄ alkyl, wherein one or more hydrogens are optionally replaced by halogen; C₁₋₅ alkylidene, wherein one or more hydrogens are optionally replaced by halogen; C₁₂₋₂₄ alkylidene, wherein one or more hydrogens are optionally replaced by halogen; C₂₋₅ alkenyl, wherein one or more hydrogens are optionally replaced by halogen; C₂₋₅ alkynyl, wherein one or more hydrogens are optionally replaced by halogen; 1-adamantyl; (1-adamantyl)methylene; C₃₋₈ cycloalkyl, wherein one or more hydrogens are optionally replaced by halogen; (C₃₋₈ cycloalkyl)methylene, wherein one or more hydrogens are optionally replaced by halogen; provided that if R³³ is C₁₋₅ alkylidene or C₁₂₋₂₄ alkylidene then the bond connecting R³³ to the ring system is a double bond, in all other above-mentioned cases said bond is a single bond. Alternatively, R³³ is a group of the following formula 2:

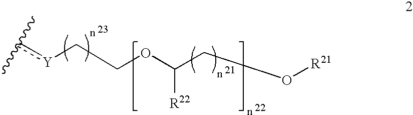

R²¹ is C₁₋₄ alkyl, preferably CH₃. Each R²² is independently H or C₁₋₃ alkyl, preferably CH₃. Y is CH₂, CH or O, provided that if Y is CH then the bond connecting Y to the ring system is a double bond, in all other cases said bond is a single bond. Preferably, Y is CH₂ or O. Each n²¹ is independently an integer of 1 or 2, preferably 1. n²² is an integer from 0 to 5, preferably from 1 to 4. If Y is O then n²³ is 1, in all other cases n²³ is 0. Preferably, R³³ is H, C₁₋₅ alkyl, C₁₋₅ alkylidene, C₁₂₋₂₄ alkyl or C₁₂₋₂₄ alkylidene. In another preferred embodiment, R³³ is the group of formula 2.

R³⁴ is H. In one preferred embodiment, R³² and R³⁴ are in a cis orientation to each other.

n³ is an integer of 1 or 2. If X is O, then n³ is preferably 1. If X is N, then n³ is preferably 2.

In accordance with the present invention it was surprisingly found that biological and/or biochemical processes involved in human diseases and disorders may be influenced by disrupting lipid rafts. This interferes with the partitioning of regulatory molecules within lipid rafts, the formation of protein complexes with lipid rafts and/or the clustering of lipid rafts, thus preventing a diseased status. Accordingly, provided herein are specific molecules, namely steroid derivatives as defined herein above which are capable of interfering with biological processes, in particular pathological processes taking place in, on, or within lipid rafts of cells, preferably diseased cells. These molecules are considered “disrafters” in accordance with this invention. Disrafters are either capable of inhibiting biosynthesis of raft components, of inhibiting or modulating the incorporation (transport) of raft components into membranes, of extracting major components of rafts from the membrane or of inhibiting interactions between raft component(s) by intercalating between them. It is also envisaged that “disrafters” are compounds which are capable ofaltering the size of lipid rafts and, thereby, inhibit (a) biological function(s) in said rafts. Accordingly, also an “augmentation” of lipid raft volume or size is considered as a disrafting process induced by the compounds provided herein. In particular, the compounds provided herein are useful in the biological process described herein above, inter alia, the prevention/inhibition of interactions between raft components by intercalation into the lipid rafts.

As documented in the appended examples the disrafting property of the compounds provided herein is determined and verified by distinct biochemical, biophysical and/or cell culture experiments. These assays comprise a disrafting liposome raftophile assay (D-LRA), a virus budding assay, a virus reproduction and infectivity assay, a degranulation assay, a SV40 infectivity assay as well as an HIV infectivity assay. The technical details are given in the appended examples.

The compounds provided herein are particularly useful in the treatment (as well as prevention and/or amelioration) of human diseases or disorders. Compounds provided herein have been scrutinized in specific biophysical/biochemical tests and have been further evaluated in cell-based disease/disorder models.

Accordingly, the compounds described herein are also useful in the treatment, prevention and/or amelioration of a disease/disorder caused by (a) biochemical/biophysical pathological process(es) occurring on, in or within lipid rafts. Corresponding examples of such diseases/disorders as well as of such biochemical/biophysical processes are given herein. The term biochemical/biophysical pathological process occurring on, in or within lipid rafts, accordingly, means for example, pathogen induced abnormal raft clustering upon viral or bacterial infections, the formation of oligomeric structures of (bacterial) toxins in lipid rafts upon infection with pathogens, or the enhanced activity of signaling molecules (like immunoglobulin E receptor) in lipid rafts. Also tighter than normal packing of lipid rafts/lipid raft components is considered a “biochemical/biophysical pathological process” in accordance with this invention.

The following compounds 10aa to 10ae are preferred examples of compound 1a.

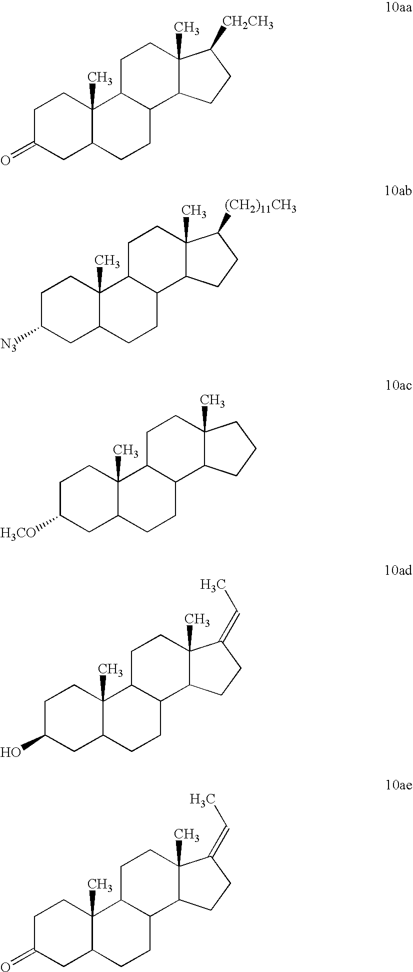

Among compounds 10aa to 10ae, compounds 10ac to 10ae are preferred.

Further preferred examples of the compound having formula 1a are the following compounds 10af to 10al:

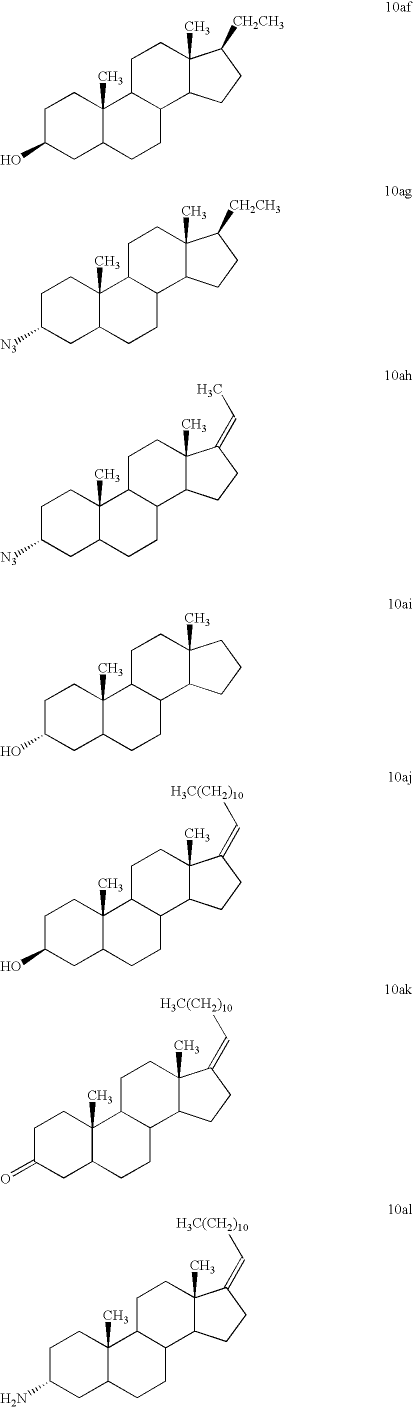

The following compounds 10ba and 10bc are preferred examples of compound 1b.

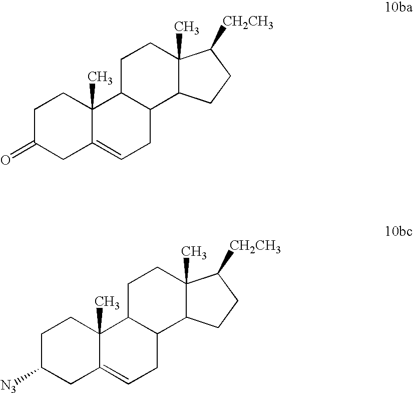

The following compound 10c is a preferred example of compound 1b.

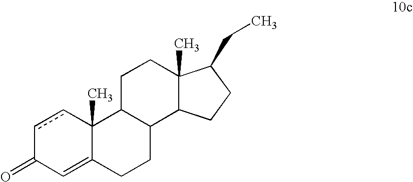

wherein [symbol] is a single bond or a double bond.

The following compounds 10da to 10dc are preferred examples of compound 1d.

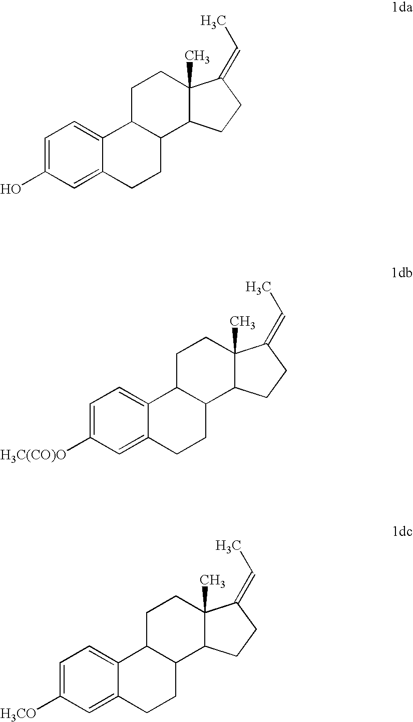

The following compounds 30a and 30b are preferred examples of compound 3.

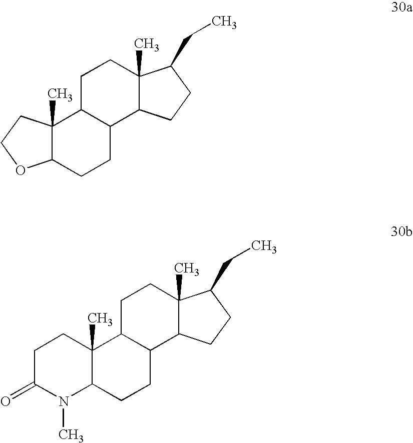

There are several structural features that impart particularly advantageous disrafting properties to steroid derivatives. These structural features can be present alone or in combination in a preferred compound.

One of these features is the deviation from the typical “flat” steroid structure by introduction of a cis ring fusion between the A and B rings of the steroid ring system. Accordingly, compounds having formula 1a in which R^{14a} is in the beta-orientation are preferred. Similarly, compounds having formula 3 in which R³² and

R³⁴ are cis to each other are preferred. Introduction of such a “kink” into the steroid ring system is believed to disturb the ordered structure of a raft upon incorporation of the kinked derivative into the raft.

A second structural feature is the presence of a bulky group in the side chain on carbon 17 of the steroid scaffold. Upon incorporation of this type of steroid derivative into the rafts, the bulky side chains are believed to disturb the raft structure. Examples for disrafters that could act via this mechanism are those steroid derivatives listed above in which R^{13a}, R^{13b}, R^{13c}, R^{13d} or R³³ are 1-adamantyl or (1-adamantyl)methylene.

Alternatively, a third structural feature is the presence of a significantly shorter, a significantly longer or no side chain on carbon 17 as compared to the side chain present on carbon 17 of cholesterol, which is a natural raft component. When incorporated into lipid rafts, such compounds bearing side chains of a length that differs from the side chain of cholesterol may pack less tightly into the raft, thus disturbing the raft structure. Examples for disrafters that could act via this mechanism are those steroid derivatives listed above in which R^{13a}, R^{13b}, R^{13c}, R^{13d} or R³³ are H, C₁₋₅ alkyl or C₁₂₋₂₄ alkyl.

A fourth structural feature is the presence of double or triple bonds in the side chain on carbon 17 of the steroid scaffold. The presence of unsaturated groups reduces the flexibility of the side chains. Upon incorporation of this type of steroid derivative into the rafts, the non-flexible side chains are believed to disturb the raft structure. Examples for disrafters that could act via this mechanism are those steroid derivatives listed above in which R^{13a}, R^{13b}, R^{13c}, R^{13d} or R³³ are C₂₋₅ alkenyl or C₂₋₅ alkynyl.

Displaying an amphiphilic side chain on carbon 17 of the steroid scaffold represents a fifth structural feature. Incorporation of such moieties into the hydrophilic sphere of rafts is believed to disturb the raft structure significantly. Examples for disrafters that could act via this mechanism are those steroid derivatives listed above in which R^{13a}, R^{13b}, R^{13c}, R^{13d} or R³³ are represented by a group of formula 2.

The presence of strong hydrogen-bond acceptors but not hydrogen-bond donors as substituents on the steroid scaffold is a sixth structural feature that can impart disrafting properties to a compound. Accordingly, compounds of formulae 1a to 1c in which R^{11a}, R^{11b} and R^{11c} are O(C₁₋₄ alkyl), N(C₁₋₄ alkyl)₂, N₃, O, S, SO₄⁻, PO₄²⁻or halogen, in particular fluorine, are a preferred subgroup. Similarly, R^{12a}, R^{12b} and R^{14b} are preferably O(C₁₋₄ alkyl), N(C₁₋₄ alkyl)₂, N₃, O or halogen, more preferably fluorine, in compounds 1a and 1b. The hydrogen-bond accepting properties of the ring heteroatoms is also a feature that is believed to impart the disrafting capability to the compounds of formula 3.

The compounds to be used in accordance with the present invention can be prepared by standard methods known in the art.

Compounds having formula 1a can be prepared from various commercially available starting materials following published synthetic protocols. Depending on the stereochemistry at position 5 of the steroid scaffold, preparation starts from either androsterone or epiandrosterone.

Various side chains of different length and structure can be easily introduced at position 17 via Wittig-type reactions (A. M. Krubiner, E. P. Oliveto, J. Org. Chem. 1966, 31, 24-26) followed by hydrogenation of the resulting double bond using hydrogen and palladium on carbon black, if a saturated side chain is intended. In contrast, unsaturation within the side chain can be realized by various common palladium-mediated couplings using suitable precursors for the Wittig process. Moreover, complete removal of the side chain can be achieved by Wolff-Kishner reduction of the 17-keto function as demonstrated in the literature (H.-J. Schneider, U. Buchheit, N. Becker, G. Schmidt, U. Siehl, J. Am. Chem. Soc. 1985, 107, 7027-7039), while side chains which have one or more oxygen atoms in the chain can be introduced via the Wittig strategy using suitable (poly)glycol precursors.

Substitution at position 3 of the steroid scaffold can be achieved by various manipulations of the 3alpha- or 3beta-hydroxy function, respectively, as outlined in various articles (A. Casimiro-Garcia, E. De Clercq, C. Pannecouque, M. Witvrouw, T. L. Stup, J. A. Turpin, R. W. Buchheit, M. Cushman, Bioorg. Med. Chem. 2000, 8, 191-200; H. Loibner, E. Zbiral, Helv. Chim. Acta 1976, 59, 2100-2113).

Various functionalities can be introduced at position 4 of the steroid scaffold by replacement of bromine in 4beta-bromoandrostane-3,17-dione, which can be prepared as described by Abul-Hajj (Y. J. Abul-Hajj, J. Org. Chem. 1986, 51, 3059-3061 and 3380-3382). On the other hand, electrophilic substituents can be introduced by trapping of the corresponding enolate. Furthermore, steroidal 4-ketones, which can be prepared along strategies described in the literature (N. L. Allinger, M. A. Darooge, R. B. Hermann, J. Org. Chem. 1961, 26, 3626-3628), can be further functionalised to obtain compounds having various substituents in position 4. Alternatively, 4-oxo-substituted steroids can be prepared using strategies outlined by Numazawa (M. Numazawa, K. Yamada, S. Nitta, C. Sasaki, K. Kidokoro, J. Med. Chem. 2001, 44, 4277-4283).

Compounds having formula 1b having a double bond at position 5 of the steroid scaffold structure can be obtained from commercially available dehydroandrosterone or dehydroepiandrosterone, respectively. The double bond can be protected as the corresponding dibromide (L. F. Fieser, Organic Syntheses, Collect. Vol. IV, Wiley, N.Y., 1963, p. 197ff). Deprotection can be achieved by debromination (D. Landini, L. Milesi, M. L. Quadri, F. Rolla, J. Org. Chem. 1984, 49, 152-153).

Compounds having formula 1c can be obtained either starting from commercially available 4-androstene-3,17-dione or by double bond isomerisation of the corresponding dehydroandrosterone derivatives. The double bond at position 1 can be introduced by oxidative processes (M. L. Lewbart, C. Mouder, W. J. Boyko, C. J. Singer, F. Iohan, J. Org. Chem. 1989, 54, 1332-1338). Alternatively, various functionalities can be introduced at position 3 by manipulation of the hydroxy group in 3beta-hydroxyandroste-4-en-17-one, which can be obtained as described in the literature (M. G. Ward, J. C. Orr, L. L. Engel, J. Org. Chem. 1965, 30, 1421-1423).

Compounds having formula 1d can be obtained from commercially available estrone. Introduction of various alkyl or alkenyl side chains at position 17 can be accomplished using a Wittig approach as previously described for other steroid examples 1a. Functional group manipulation at position 3 can be achieved via transformation of the hydroxy functional group of estrone into a leaving group, e.g. a nonaflate, and subsequent transition metal-mediated cross-coupling reactions (M. Rottländer, P. Knochel, J. Org. Chem. 1998, 63, 203-208; X. Zhang, Z. Sui, Tetrahedron Lett. 2003, 44, 3071-3073) or by simple alkylation or acylation.

Azasteroid derivatives having formula 3 (i.e. X is N) can be prepared as described in the literature (G. H. Rasmusson, G. F. Reynolds, N. G. Steinberg, E. Walton, G. F. Patel, T. Liang, M. A. Cascieri, A. H. Cheung, J. R. Brooks, C. Berman, J. Med. Chem. 1986, 29, 2298-2315, and literature cited therein; N. J. Doorenbos, C. L. Huang, J. Org. Chem. 1961, 26, 4548-4550).

Synthetic access to oxasteroids having formula 3 (i.e. X is 0) can be achieved by strategies described by Doorenbos and others (N. J. Doorenbos, M. T. Wu, J. Org. Chem. 1961, 26, 4550-4552; R. B. Turner, J. Am. Chem. Soc. 1950, 72, 579-585; G. R. Pettit, T. R. Kasturi, J. Org. Chem. 1961, 26, 4557-4563; H. Suginome, S. Yamada, Bull. Chem. Soc. Jpn. 1987, 60, 2453-2461, and literature cited therein) and combinations thereof with strategies described for compounds having formulae 1a, 1b, 1c and 1d.

Starting from commercially available epiandrosterone, compound 10aa can be prepared by Wittig reaction with ethylidenetriphenylphosphorane (A. M. Krubiner, E. P. Oliveto, J. Org. Chem. 1966, 31, 24-26) and subsequent hydrogenation of the double bond, followed by pyridinium chlorochromate oxidation of the 3-hydroxy function.

Starting from commercially available epiandrosterone, compound 10ab can be prepared by Wittig reaction with 1-dodecylidenetriphenylphosphorane, followed by hydrogenation, formation of the 3beta-mesylate and substitution of the mesyl group by azide (A. Casimiro-Garcia, E. De Clercq, C. Pannecouque, M. Witvrouw, T. L. Stup, J. A. Turpin, R. W. Buckheit, M. Cushman, Bioorg. Med. Chem. 2000, 8, 191-200).

O-methylation of commercially available androsterone by treatment with sodium hydride and methyl iodide, followed by Wolff-Kishner reduction of the 17-keto function (H.-J. Schneider, U. Buchheit, N. Becker, G: Schmidt, U. Siehl, J. Am. Chem. Soc. 1985, 107, 7027-7039) can provide compound 10ac.

Compound 10ad can be prepared from commercially available epiandrosterone as described in the literature (A. M. Krubiner, E. P. Oliveto, J. Org. Chem. 1966, 31, 24-26). Subsequent oxidation using pyridinium chlorochromate can afford 10ae.

Compound 10af can be prepared from compound 10ad by hydrogenation of the double bond using hydrogen and palladium on charcoal.

Compound 10ag can be obtained from 10af by reaction with mesyl chloride and subsequent substitution of the mesylate by azide.

Compound 10ah can be prepared using the same strategy as outlined for 10ag, but starting from 10ad as substrate.

Compound 10ai can be derived from commercially available androsterone by treatment with p-toluenesulfonhydrazide and sodium borohydride in a Wolff-Kishner-type reduction of the 17-keto function to methylene (L. Caglioti, Organic Syntheses 1972, 52,122-124).

Compound 10aj can be prepared from epiandrosterone via the above described Wittig strategy using commercially available dodecyltriphenylphosphonium bromide as a reagent.

Compound 10ak can be obtained from 10aj employing a simple pyridinium chlorochromate mediated oxidation.

Compound 10al can be prepared from 10aj via the corresponding mesylate, which is replaced by azide followed by reduction to the corresponding amine with lithium aluminum hydride.

Bromination at positions 5 and 6 of commercially available dehydroepiandrosterone (L. F. Fieser, Organic Syntheses, Collect. Vol. IV, Wiley, N.Y., 1963, p. 197), followed by Wittig reaction and hydrogenation as previously described, followed by treatment of the so-obtained product with excess sodium borohydride (Y. Houminer, J. Org. Chem. 1975, 40, 1361-1362) to restore the 5,6-double bond can provide compound 10ba.

The 17beta-ethyl-3beta-hydroxy substituted dibromide used as an intermediate in the preparation of 10ba can be transformed into the corresponding mesylate, followed by substituion with azide and debromination as described in the literature (D. Landini, L. Milesi, M. L. Quadri, F. Rolla, J. Org. Chem. 1984, 49, 152-153) to give compound 10bc. The same intermediate can be used in the synthesis of compounds having formula 10c. Debromination (Y. Houminer, J. Org. Chem. 1975, 40, 1361-1362), pyridinium chlorochromate oxidation of the 3-hydroxy function, followed by acid-mediated isomerisation of the double bond to connect positions 4 and 5 can provide compound 10c, in which positions 1 and 2 are connected by a single bond. This bond can be converted into a double bond by dichlorodicyanoquinone oxidation (M. L. Lewbart, C. Mouder, W. J. Boyko, C. J. Singer, F. Iohan, J. Org. Chem. 1989, 54, 1332-1338).

Compound 10da can be prepared by treatment of estrone with commercially available ethyltriphenylphosphonium iodide under standard Wittig conditions.

Acylation of 10da with acetanhydride and DMAP and methylation with methyl iodide provide compounds 10db and 10dc, respectively.

Compound 30a can be synthesized as outlined by Suginome (H. Suginome, Y. Shinji, Bull. Chem. Soc. Jpn. 1987, 60, 2453-2461) starting from compound 10c, which can be prepared as described above.

The heterocyclic A ring of compound 30b can be prepared as described in the literature (N. J. Doorenbos, C. L. Huang, J. Org. Chem. 1961, 26, 4548-4550). A beta-ethyl side chain at position 17 of the steroid scaffold, which is required in the substrate for this strategy, can be introduced starting from epiandrosterone as described above.

In accordance with the data and information provided herein the present invention provides in particular for the use of the compounds as shown in formulae 10ac, 10ad, 10ae, 10af, 10ag, 10ak, 10al, 10da, 10db and 10dc in a medical setting for the treatment of human as well as animal disorders and diseases which are characterized by biological processes taking place in or on lipid rafts. As will be detailed herein below, these diseases and/or disorders comprise, for example neurodegenerative disorders like Alzheimer's disease or prion-related diseases/disorders, Creutzfeldt-Jakob disease, Kuru, Gerstmann-Sträussler-Scheinker syndrome and fatal familial insomnia (FFI) as well as infectious diseases like viral, bacterial or parasite infections. Furthermore and as documented in the appended examples immunological and/or allergic disorders may be ameliorated, prevented or treated by the compounds provided herein. These disorders comprise, in particular hyperallergenic disorders (asthma), autoimmune diseases (like Batten disease), systemic lupus erythematosus or arteriosclerosis. Further disorders like proliferative disorders (cancer) and systemic disorders like diabetes are considered valuable targets to be treated by the compounds provided herein. Of particular interest in this context are, however, infectious diseases (preferably viral and bacterial diseases, most preferably influenza infections) as well as the immunological or hyperallergenic disorders, like asthma.

Prior to investigating the inhibitory activity of compounds given in the present invention in various biological assays, said compounds may also be evaluated in several toxicity assays in order to document their safety in the concentration range used or to determine their highest non-toxic concentration. Thus, it can be assured that observed inhibitory effects in each disease-relevant assay are not due to toxic effects exerted by the compound under evaluation. Toxicity assays are well known in the art and may, inter alia, comprise lactate dehydrogenase (LDH) or adenylate kinase (AK) assays or an apoptosis assay. Yet, these (cyto)-toxicity assays are, as known by the skilled artisan, not limited to these assays. The following assays are, accordingly, non-limiting examples.

The release of lactate dehydrogenase (LDH) from cultured cells exposed to a substance provides a sensitive and accurate marker for cellular toxicity in routine biocompatibility testing in vitro (Allen, Promega Notes Magazine 45 (1994), 7). Promega's commercial CytoTox-ONE™ LDH assay kit (Promega # G7891) represents a homogeneous membrane integrity assay combined with a fluorometric method for estimating the number of nonviable cells present in multiwell plates.

The assay may be performed according to the manufacturer's instructions (Promega Technical Bulletin No. 306) in triplicate wells for each compound concentration. The incubation period is 16h for MDCK cells and 1.5h for RBL cells, corresponding to the exposure time in the assays for which the LDH assay serves as reference (focus reduction assay and degranulation assay). Solvent controls may be done only at the highest solvent concentration.

A maximum assay readout can be provided by adding detergent to three wells of the 96-well plate (as decribed in the Promega protocol). The background can consist of wells without cells. Each well may be processed and calculated independently, so that each plate contains the necessary controls. Triplicate readings are averaged, the average background subtracted and the resulting value converted to % maximum. A threshold of toxicity may be defined as follows: for MDCK cells the threshold may be defined as twice the percentage of untreated or solvent-treated controls.

If the result at a certain compound concentration is below threshold, this concentration may be deemed non-toxic. The highest non-toxic concentration, the maximal tolerated concentration, dose, may be defined as the highest dose at which toxicity was not observed.

All evaluations of compounds in assays described herein can be processed at the maximal tolerated concentration as determined in the LDH release assay or below.

In a second assay, the release of the enzyme adenylate kinase (AK) from damaged cells is measured. AK, a robust protein present in all eukaryotic cells, is released into the culture medium when cells die. The enzyme phosphorylates ADP to generate ATP, which is measured using the bioluminescent firefly luciferase reaction.

After 18h and 48h incubation time 20μL of the supernatant of each well is transferred into new plates and the ToxiLight assay (Cambrex) is performed according to the manufacturer's instructions (ToxiLight, Cambrex Bio Science, Rockland, USA, cat# LT07-117). After the conversion of added ADP to ATP by the adenylate kinase, luciferase catalyses the formation of light from ATP and luciferin in a second step. The luminescence measurements are performed with a Genios Pro instrument (TECAN).

This assay may be performed prior to the SV40 assay described in the experimental part in order to confirm that observed inhibition is not due to compound-induced damage of the cells.

In a third assay, the induction of apoptosis exerted by the compounds provided in the present invention is evaluated. Loss of the phospholipid asymmetry of the cell membrane represents one of the earliest cellular changes of the apoptotic process (Creutz, C. E. (1992) Science 258, 924). Annexins are ubiquitous homologous proteins that bind phospholipids in the presence of calcium. As the movement of phosphatidylserine from the internal leaflet to the external leaflet of the phospholipids bilayer represents an early indicator of apoptosis, annexin V and its dye conjugates can be used for the detection of apoptosis because they interact strongly and specifically with the exposed phosphatidylserine (Vermes (1995) J. Immunol. Methods 184, 39).

The assay may be performed according the manufacturer's instructions (Annexin V Conjugates for Apoptosis Detection, Molecular Probes, cat# A13201). After 72h incubation time DRAQ5™ is added to the cells at a final concentration of 5 μM. After 1h incubation time the medium was discarded and AnnexinV conjugated to Alexa Fluor 488 (Alexa488; Molecular Probes) is added (250 ng per mL). After incubation and washing, the cells are fixed with paraformaldehyde and a microscopic analysis with an OPERA automated confocal fluorescence microscope (Evotec Technologies GmbH) is performed using 488 and 633 nm laser excitation and a water-immersion 10-fold objective. Four images per well can be taken automatically, the total number of cells (DRAQ5) and the area of AnnexinV-Alexa488 can be determined by automated image analysis and average and standard deviations for triplicates may be calculated. The apoptotic index can be calculated by dividing the area of AnnexinV (pixels) with the total number of nuclei (DRAQ5 stained), multiplied by 100%. The result can be expressed as a comparison to untreated cells after normalization to the background (solvent-treated cells).

This assay can also be performed prior to the SV40 assay described below in order to confirm that observed inhibition is not a consequence of the induction of apoptosis subsequent to compound addition.

Finally, by visual evaluation of cell morphology during assay operation using a light microscope evidence of toxic effects caused by the tested compounds can be assessed.

In the following more detailed information on diseases and disorders are given. These diseases and disorders may be prevented, ameliorated or treated by using the compounds provided herein. Without being bound by theory, in some cases mechanistic models are given how the compounds described herein may function. Compounds provided herein are particularly useful in this medical context, whereas particularly preferred compounds are the compounds shown in formulae 10ac, 10ad, 10ae, 10af, 10ag, 10ak, 10al, 10da, 10db and 10dc. In particular, the experimental data provided herein document that 10ac, 10ad, 10ae, 10af, 10ag, 10ak, 10al, 10da, 10db and 10dc are particularly preferred compounds in distinct medical interventions or preventions.

Alzheimer disease (AD) depends on the formation of amyloid plaques containing the amyloid-beta-peptide (Aβ), a fragment derived from the large type I transmembrane protein APP, the amyloid precursor protein. The Aβ fragment is cleaved sequentially by enzymes termed beta-secretase (BACE) and gamma-secretase. BACE is an aspartyl-protease that cleaves APP in its luminal domain, generating a secreted ectodomain. The resulting 10-kDa C-terminal fragment is subsequently cleaved by gamma-secretase, which acts at the transmembrane domain of APP, thus releasing Aβ. A third enzymatic activity, the alpha-secretase, counteracts the activity of BACE by cleaving APP in the middle of the Aβ region, yielding products that are non-amyloidogenic: The beta fragment (a secreted ectodomain) and the short C-terminal stub that is also cleaved by beta-secretase. Therefore, alpha-cleavage directly competes with beta-cleavage for their common substrate APP. Lipid rafts play a role in regulating the access of beta-secretase to the substrate APP. The compounds provided herein are supposed to disrupt lipid rafts and, thereby to inhibit beta-secretase cleavage. Without being bound by theory, this may be achieved either by 1) interfering with the partitioning of APP and BACE in rafts, 2) the intracellular trafficking of APP and BACE to meet within the same rafts and 3) the activity of BACE in rafts, to inhibit Aβ fragment production and generation of Alzheimer disease.

Steroid derivatives as disclosed herein will align with and bind non-covalently to raft constituents, especially sphingosine and ceramide derivatives. Without being bound by theory, this is likely to cause an expansion and disordering of the phase and inhibition of enzymatic e.g. beta-secretase, and other activities dependent upon an ordered lipid phase. Thus steroidal derivatives disclosed herein are useful as pharmaceuticals for neurodegenerative diseases e.g. Alzheimer's disease (beta-secretase inhibition); Creutzfeldt-Jakob disease (inhibition of prion protein processing and amyloid formation).

Also prion disorders may be treated and/or ameliorated by the medical use of the compound provided herein. A conformational change resulting in amyloid formation is also involved in the pathogenesis of prion disease. Prion diseases are thought be promoted by an abnormal form (PrPsc) of a host-encoded protein (PrPc). PrPsc can interact with its normal counterpart PrPc and change the conformation of PrPc so that the protein turns into PrPsc. PrPsc then self-aggregates in the brain, and these aggregates are thought to cause the disorders manifested in humans as Creutzfeldt-Jakob disease, Kuru, Gerstmann-Sträussler-Scheinker syndrome, or fatal familial insomnia (McConnell, Annu. Rev. Biophys. Biomol. Struct. 32 (2003), 469-492). The mechanism by which PrPc is converted to PrPsc may involve lipid rafts. PrP is a GPI-anchored protein. Both PrPc and PrPsc are associated with DRMs in a cholesterol-dependent manner. The GPI anchor is required for conversion. When the GPI anchor is replaced by a transmembrane domain, conversion to abnormal proteins is blocked. In vitro, the conversion of PrPc to PrPsc, as monitored by PrP protease resistance, occurs when microsomes containing PrPsc are fused with DRMs containing PrP (Baron (2003) J. Biol. Chem. 278, 14883-14892; Stewart (2003) J. Biol. Chem. 278, 45960-45968). Extraction with detergent leads to raft clustering in DRMs. Fusion of microsomes with DRMs was necessary in this experiment because simply mixing the membranes did not lead to measurable generation of new PrPsc.

Lipid rafts promote, accordingly, abnormal prion conversion. Endocytosis has also been shown to play a role for prion conversion, as is the case for BACE cleavage of APP. Rafts containing PrPc and PrPsc probably become clustered after endocytosis. It is also possible that the protein factor X, postulated to mediate conversion, is involved in raft clustering after endocytosis. If PrPc and PrPsc were clustered into the same raft platform after endocytosis, an increase of interaction efficiency would result and lead to amplification of conversion. Accordingly, the compounds of the invention are also useful in the treatment and/or prevention of prion diseases.

Several viruses and bacteria employ lipid rafts to infect host cells. In particular, lipid rafts are involved in the entry, assembly and egress of several enveloped viruses. As shown in the appended technical examples, influenza virus is a prototype of such a virus.

The compounds described in this invention (disrafters) can be applied to 1) disrupt rafts and interfere with the transport of hemagglutinin and neuraminidase to the cell surface, 2) prevents the clustering induced by M proteins of rafts containing the spike glycoproteins induced by M proteins and, thus, interfere with virus assembly, or 3) by increasing the size/volume of lipid rafts or 4) prevent the fission of the budding pore (pinching-off) which occurs at the phase border of raft (viral membrane) and non-raft (plasma membrane). Particularly preferred compounds in this regard are compounds 10ae and 10af, and compounds 10ad and 10al represent an even more preferred embodiment within the context of the present invention. Corresponding experimental evidence is provided in the appended examples. It is of note that also further data, e.g. provided in the SV40 assay described herein, showed good inhibitory effects, in particular compounds 10ac, 10ad, 10af as well as 10db.

In viral infection, raft clustering is involved in the virus assembly process. The steroidal derivatives 10ad, 10ae, 10af and 10al disrupt the lipid ordered structure by augmentation (see assay descriptions). They also have an effect in a virus replication assay. Without being bound by theory, the structural feature underlying this effect is thought to be represented by the combination of a polar 3-substitution inside the steroidal A ring and the presence of a lipophilic alkyl or alkylidene substituent at position 17 comprising, for example, a two carbon unit as in 10ad, 10ae and 10af or a twelve carbon unit as in 10al. Using an 3α-amino group as polar function in the A-ring results in increased potency of compound 10al, thus indicating the 3α-amino substitution pattern as an even more preferred embodiment. As demonstrated by the results obtained in the viral replication assay provided in the experimental part, these compounds may be useful for pharmaceutical intervention. In contrast androsterone, epiandrosterone and cholesterol do not show significant disrafting activity nor are they effective in a model assay for influenza infection.

As the mechanism of virus release for HIV-1 is similar to that of influenza virus, with respect to raft involvement, the above compounds can also be developed for the treatment of AIDS. To demonstrate this, compounds were tested for inhibition of infection of HeLa TZM cells by the HIV-1 strain NL4-3 (laboratory adapted B-type strain) as a disease model for AIDS. Particularly preferred compounds in this context are 10ak, 10da and 10db, and the compound represented by formula 10dc provides an even more preferred substance for the pharmaceutical intervention in the case of HIV infection. Corresponding evidence is provided in the experimental part.

Further viral diseases (as non limiting examples) which may be approached with the above compounds or derivatives thereof are herpes, ebola, enterovirus, Coxsackie virus, hepatitis C, rotavirus and respiratory syncytial virus. Accordingly, particularly preferred compounds as well as preferred compounds provided herein in the context of a specific (viral) assay or test system may also be considered useful in the medical intervention and/or prevention of other infectious deseases, in particular viral infections.

As detailed herein, the compounds which are active in the disruption of lipid rafts in cells infected with influenza virus or in the SV40 assay may also be employed in other medical settings, in particular in other viral infection, most preferably in HIV infections. It is also envisaged that compounds shown to be useful in AIDS intervention/HIV infection are of use in further infectious diseases, like other viral infections.

Herpes simplex virus (HSV) entry requires the interaction of viral glycoproteins with a cellular receptor such as herpesvirus entry mediator (HVEM or HveA) or nectin-1 (HveC). During HSV infection, a fraction of viral glycoprotein gB associates with lipid rafts, as revealed by the presence in detergent-resistant membranes (DRM). Disruption of lipid rafts via cholesterol depletion inhibits HSV infection, suggesting that HSV uses lipid rafts as a platform for entry and cell signalling (Bender). The rafts-disrupting agents of the invention may be employed in the inhibition of the partitioning of either viral glycoproteins or an interacting molecule into rafts as a strategy to inhibit infection and replication of HSV.

Also Ebola virus assembly and budding depends on lipid rafts. These functions depend on the matrix protein VP40 that forms oligomers in lipid rafts. The use of compounds described in this invention leads to a disruption of lipid rafts. This may be used as a means to inhibit VP40 oligomerization and, consequently, Ebola virus infection and assembly.

Enteroviruses use the complement regulatory protein decay-accelerating factor (DAF), a GPI-anchored protein, as a receptor to infect cells. Like other GPI-anchored proteins, DAF partitions to lipid rafts. Consistently, viruses infecting the cell via this receptor system depend on lipid rafts. In particular, lipid rafts appear to be essential for virus entry, after binding to the cell surface. Furthermore, viruses using the DAF receptor system copurify with lipid raft components in a DRM extraction assay. Since lipid rafts enable enteroviruses to enter cells, compounds as disclosed in this invention that disrupt lipid rafts or the partitioning of DAF to lipid rafts or the post-binding events leading to cell infection, can be used for the prevention and treatment of enterovirus-based disorders.

Coxsackie virus entry and cell infection depend on lipid rafts. Receptor molecules (integrin αvβ3 and GRP78) accumulate in lipid rafts following Coxsackie virus infection. The raft-disrupting compounds of the invention disrupt lipid rafts or the partitioning of Coxsackie virus receptors to lipid rafts or the post-binding events leading to cell infection and may, accordingly, be used for the prevention and treatment of Coxsackie virus-based disorders (as well as in disorders caused by viruses, similar to Coxsackie virus.

Rafts are also implicated in the life cycle of Human Immunodeficiency Virus (HIV) and, accordingly, in AIDS. Without being bound by theory, disrafters of the present invention can be applied to disrupt rafts and interfere with the transport of HIV glycoproteins to the cell surface, prevent the clustering of rafts containing the spike glycoproteins induced by Gag proteins and, thus, interfere with virus assembly. Accordingly, the compounds described herein are also medically useful in the treatment and amelioration of HIV-infections and AIDS. As mentioned herein above, preferred compounds in this context are compounds which are qualified as “disrafters” in accordance with this invention and which show positive results in the appended “influenza assay” which is an assay for testing the efficacy of a compound described herein. Compounds which show positive results in the appended “influenza assay”, may, accordingly, also be employed in the treatment, prevention and/or amelioration of other vial infections, like HIV-infections (e.g. AIDS).

Lipid rafts are also involved in the infectious cycle of hepatitis C virus (HCV). The compounds described in this invention as “disrafters” may disrupt lipid rafts or the partitioning of proteins constituents of viral replication complex to lipid rafts or interfere with the replication events leading to virus assembly. Accordingly, the compounds described herein are also useful in the prevention and treatment of hepatitis, in particular of hepatitis C.

Rotavirus cell entry depends on lipid rafts. Molecules implicated as rotavirus receptors such as ganglioside GM1, integrin subunits α2β3, and the heat shock cognate protein 70 (hsc70) are associated with lipid rafts. Furthermore, rotavirus infectious particles associate with rafts during replication and lipid rafts are exploited for transport to the cell surface. The compounds described herein may be employed to disrupt lipid rafts or the partitioning of receptors for Rotavirus, the formation of protein and lipids complexes necessary for replication and transport via lipid rafts. Accordingly, they are useful in the prevention and treatment of Rotavirus infection.

Simian virus 40 (SV40) enters cells via an atypical caveolae-mediated endocytic pathway rather than via clathrin-coated pits, (Anderson (1996) Mol. Biol. Cell 7, 1825-1834; Stang (1997) Mol. Biol. Cell 8, 47-57). This mechanism of cellular uptake is also employed by members of the virus family Coronaviridae, which are the responsible pathogens causing human diseases such as severe acute respiratory syndrome (SARS) and upper respiratory tract infections, and by the respiratory syncytial virus (Macnaughton (1980) J. Clin. Microbiol. 12, 462-468; Nomura (2004) J. Virol. 78, 8701-8708; Drosten (2003) N. Engl. J. Med. 348, 1967-1976; Ksiazek (2003) N. Engl. J. Med. 348,1953-1966). Moreover, bacteria also use this mechanism for cellular uptake, e.g. Mycobacterium spp. which cause tuberculosis. Thus, the herein presented SV40 assay serves as model for caveolae-mediated cellular uptake, and the compounds described in the present invention may be used for pharmaceutical intervention in the case of diseases caused by the above described viruses and bacteria.

Uptake of Simian Virus 40 (SV40) into caveolae rafts is a model for infection by diverse bacteria and viruses which utilize the raft to gain entry to the cell (Pelkmans (2002) Science 296, 535-539). The assay is used as a screen for compounds which may inhibit bacterial or viral infection at the stage of caveolar incorporation, endocytosis and early intracellular trafficking. This mechanism is particularly relevant to infection by respiratory syncytial virus, coronavirus (e.g. SARS) and to bacterial infection by Mycobacterium spp., leading to tuberculosis. Accordingly, compounds which show positive results in the appended SV40 assay may also be used in the context of medical intervention of infections of the respiratory tract, like tuberculosis and bacterial infestation by, but not limited to, Campylobacter spp., Legionella spp., Brucella spp., Salmonella spp., Shigella spp., Chlamydia spp., FimH and Dr⁺ Escherichia coli.

The compounds presented herein are suitable to inhibit such uptake by a caveolae-mediated mechanism as demonstrated by the SV40 assay using HeLa cells infected with wild type SV40 viruses. Moreover, the lack of inhibition in a similar assay using Vesicular Stomatitis Virus (VSV) demonstrates the capability of this working hypothesis, as VSV enters via clathrin-mediated endocytosis into early and late endosomes. In this context, compounds 10ac, 10ad and 10af are particularly preferred substances for the treatment of given infections. Moreover, compound 10db represents an even more preferred embodiment for the pharmaceutical intervention in the case of viral and/or bacterial infections.

As pointed out above, the compounds described herein may also be employed in the treatment or amelioration of bacterial infections and toxicoses induced by secreted bacterial toxins.

Bacterial toxins such as cholera (from Vibrio cholerae), aerolysin (Aeromonas hydrophilia), anthrax (Bacillus anthracis) and helicobacter toxin form oligomeric structures in the raft, crucial to their function. The raft is targeted by binding to raft lipids such as ganglioside GM1 for cholera. Prevention of oligomerization is equivalent to prevention of raft clustering, hence the same or similar compounds as those used for viral infection should be able to inhibit the activity of bacterial toxins. However, a difference in dosing regimen would be expected as toxins will be rapidly cleared from the blood and treatment may be short in comparison to viral infection where a course of treatment may be necessary.

In bacterial infection such as tuberculosis, shigellosis and infection by Chlamydia and uropathogenic bacteria the organism is taken up into the cell in a raft-dependent internalization process often involving caveolae. Prevention of localization of the bacterial receptor in rafts or blockage of internalization would prevent infection. In the case of caveolae, which depend on a cholesterol binding protein, caveolin, exclusion of cholesterol from the raft with steroid derivatives may prevent uptake of the pathogen.

Tuberculosis is an example of a bacterial infectious disease involving rafts. First, Complement receptor type 3 (CR3) is a receptor able to internalize zymosan and C3bi-coated particles and is responsible for the non-opsonic phagocytosis of Mycobacterium kansasii in human neutrophils. In these cells CR3 has been found associated with several GPI-anchored proteins localized in lipid rafts of the plasma membrane. Cholesterol depletion markedly inhibits phagocytosis of M. kansasii, without affecting phagocytosis of zymosan or serum-opsonized M. kansasii. CR3, when associated with a GPI protein, relocates in cholesterol-rich domains where M. kansasii are internalized. When CR3 is not associated with a GPI protein, it remains outside of these domains and mediates phagocytosis of zymosan and opsonized particles, but not of M. kansasii isopentenyl pyrophosphate (IPP), a mycobacterial antigen that specifically stimulates Vgamma9Vdelta2 T cells. Accordingly, the present invention also provides for the use of the compounds disclosed herein in the treatment and/or amelioration of an Mycobacterium infection, preferably of a Mycobacterium tuberculosis infection.

Shigellosis is an acute inflammatory disease caused by the enterobacterium Shigella. During infection, a molecular complex is formed involving the host protein CD44, the hyaluronan receptor, and the Shigella invasin IpaB, which partitions during infection within lipid rafts. Since raft-dependent interactions of host cellular as well as viral proteins are required for the invasion process, the compounds described herein may be employed to disrupt lipid rafts or the partitioning of receptors for Shigella, the partitioning of Shigella proteins, the formation of protein and lipids complexes necessary for replication and transport via lipid rafts. Therefore, the invention also provides for the medical/pharmaceutical use of the compounds described herein the treatment or amelioration of shigellosis.

Chlamydia pneumoniae, an important cause of respiratory infections in humans that additionally is associated with cardiovascular disease, Chlamydia psittaci, an important pathogen in domestic mammals and birds that also infects humans, as well as other Chlamydia strains (C. trachomatis serovars E and F), each enter host cells via lipid rafts.

The compounds of the invention may be used to disrupt lipid rafts or the partitioning of protein and lipids complexes necessary for replication and transport via lipid rafts, can be used for the prevention and treatment of Chlamydia infection, in particular C. pneumonia infections.

Type 1 fimbriated Escherichia coli represents the most common human uropathogen, that invades the uroepithelium despite its impermeable structure, via lipid rafts-dependent mechanisms. The compounds provided herein may disrupt lipid rafts or caveolae, the partitioning of protein and lipids complexes necessary for the binding of E. coli, transport via lipid rafts and subsequent infection across the bladder and similar epithelia. Therefore, the compounds described in the invention may be used for the prevention and treatment of bacterial infectious diseases, in particular uropathologies.

Various bacterial toxins exploit rafts to exert their cytotoxic activity. For example, the pore-forming toxin aerolysin, produced by Aeromonas hydrophila, on mammalian cells binds to an 80-kD glycosyl-phosphatidylinositol (GPI)-anchored protein on BHK cells and partitions in rafts. The protoxin is then processed to its mature form by host cell proteases. The preferential association of the toxin with lipid rafts increases the local toxin concentration and thereby promotes oligomerization, a step that it is a prerequisite for channel formation. Accordingly, the compounds described herein are also useful in the treatment, prevention or amelioration of a disease related to a bacterial infection. In context of this invention, it is also envisaged that the compounds described herein are employed in co-therapy approaches. Accordingly, it is also envisaged that the compounds are administered to a patient in need of treatment in combination with further drugs, e.g. antibiotics.

The protective antigen (PA) of the anthrax toxin binds to a cell surface receptor and thereby allows lethal factor (LF) to be taken up and exert its toxic effect in the cytoplasm. Clustering of the anthrax toxin receptor (ATR) with heptameric PA or with an antibody sandwich causes its association to specialized cholesterol and glycosphingolipid-rich microdomains of the plasma membrane (lipid rafts). Altering raft integrity using drugs prevented LF delivery and cleavage of cytosolic MAPK kinases.

“Disrafters” as disclosed herein may be applied to disrupt rafts and interfere with the clustering/oligomerization of toxins. Accordingly, the compounds of the invention are also useful in the treatment/prevention of an infection with Bacillus anthracis.

Helicobacter pylori has been implicated in the generation of chronic gastritis, peptic ulcer, and gastric cancer. Lipid rafts play a role in the pathogenetic mechanisms of Helicobacter pylori intoxication. Therefore, the compounds described herein are also useful in the treatment, prevention or amelioration of a Helicobacter infection, e.g. the treatment of gastritis, peptic ulcers and/or gastric ulcers.

The compounds described herein are also useful in the treatment/prevention of an infection with plasmodium, in particular P. falciparum. Accordingly, the compounds described herein may be employed to disrupt lipid rafts or caveolae, the partitioning of protein and lipids complexes necessary for the binding of Plasmodium falciparum to red blood cells, or the transport via lipid rafts and subsequent infection. Therefore, they may be used for the prevention and treatment of malaria.

Also asthma and other immunological diseases may be treated by the use of the compounds as disclosed herein. The cells used most intensively to study the role of lipid rafts in FcεRI-mediated signaling are rat basophilic leukemia (RBL) cells. A role for rafts in the interactions that follow FcεRI aggregation, mainly in signaling complexes assembled around the linker for activation of T cells (LAT), is described (Metzger, Mol. Immunol. 38 (2002), 1207-1211).

The compounds as described herein may be applied to disrupt rafts and 1) interfere with the transport and aggregation of FcεRI at the cell surface, 2) interfere with the transport and aggregation of rafts by LAT at the cell surface. Accordingly, the invention also provides for the use of the compounds disclosed herein in the treatment/prevention of asthma. The compounds described herein provide positive results in a cell based assay (degranulation assay) which is an assay for testing substances useful in immunological as well as auto-immunological disorders.

A particularly preferred compound for such treatment is compound 10al which inhibits the release of β-hexosaminidase used as marker in the degranulation assay efficiently. Thus, as exemplified with compound 10al, the combination of a long, lipophilic 17-dodecylidene side chain with a polar 3α-amino function inside the A-ring represents a preferred substitution pattern for the pharmaceutical intervention in the case of immunological diseases, in particular asthma.

Accordingly, also autoimmune diseases as well as hyperallergic responses may be treated by the compounds/disrafters disclosed herein.

Neuronal ceroid lipofuscinoses, also termed Batten disease, are a heterogeneous group of autosomal recessively inherited disorders causing progressive neurological failure, mental deterioration, seizures and visual loss secondary to retinal dystrophy. The juvenile type is of special interest to the ophthalmologist as visual loss is the earliest symptom of the disorder. This occurs as a result of mutations in the CLN3 gene, encoding a putative transmembrane protein CLN3P, with no known function. CLN3P resides on lipid rafts. Therefore, the compounds described herein are useful in the treatment of, e.g., Batten disease.

Systemic lupus erythematosus (SLE) is characterized by abnormalities in T lymphocyte receptor-mediated signal transduction pathways. Lymphocyte-specific protein tyrosine kinase (LCK) is reduced in T lymphocytes from patients with SLE and this reduction is associated with disease activity. Molecules that regulate LCK homeostasis, such as CD45, C-terminal Src kinase (CSK), and c-Cbl, are localized in lipid rafts. Therefore, also SLE is a medical target for the use of the compounds disclosed in this invention.

In a further embodiment of the invention, atherosclerosis is to be treated/ameliorated or even prevented by the use of the compounds described herein in medical settings and/or for the preparation of a pharmaceutical composition.

Also proliferative disorders, like cancers may be targeted by the compounds described herein. A large number of signaling components are regulated through their partitioning to rafts. For example, the tyrosine kinase activity of EGF receptor is suppressed in rafts and cholesterol plays a regulatory role in this process. Similarly, H—Ras is inactive in rafts and its signaling activity occurs upon exiting rafts. Rafts have also been shown to play a role in the regulation of apoptosis. Disrafters/compounds disclosed herein may be used in the treatment of cancer, e.g. the treatment of leukemias or tumorous diseases, as well as melanomas.

A further interventional opportunity is to prevent mitogenic receptor signaling. As for immunogenic signaling, this involves the establishment of a raft based signaling platform for a ligand activated receptor. It would be expected that similar molecules to those described for immunoglobulin E receptor signaling would also inhibit mitogenic signaling.

Insulin signalling leading to GLUT-4 translocation depends on insulin receptor signalling emanating from caveolae or lipid rafts at the plasma membrane. Accordingly, in a further embodiment of the invention, the compounds described herein may be used in the preparation of a pharmaceutical composition for the treatment of insulin-related disorders, like a systemic disorder, e.g. diabetes.

The compounds described in this invention are particularly useful in medical settings, e.g. for the preparation of pharmaceutical composition and the treatment, amelioration and/or prevention of human or animal diseases. The patient to be treated with such a pharmaceutical composition is preferably a human patient.

The compounds described as “disrafters” herein may be administered as compounds per se in their use as pharmacophores or pharmaceutical compositions or may be formulated as medicaments. Within the scope of the present invention are pharmaceutical compositions comprising as an active ingredient a compound of one of the formulae 1a, 1b, 1c and 1d defined above. The pharmaceutical compositions may optionally comprise pharmaceutically acceptable excipients, such as carriers, diluents, fillers, desintegrants, lubricating agents, binders, colorants, pigments, stabilizers, preservatives or antioxidants.

The pharmaceutical compositions can be formulated by techniques known to the person skilled in the art, such as the techniques published in Remington's Pharmaceutical Sciences, 20^{th} Edition. The pharmaceutical compositions can be formulated as dosage forms for oral, parenteral, such as intramuscular, intravenous, subcutaneous, infradermal, intraarterial, rectal, nasal, topical or vaginal administration. Dosage forms for oral administration include coated and uncoated tablets, soft gelatine capsules, hard gelatine capsules, lozenges, troches, solutions, emulsions, suspensions, syrups, elixiers, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insuflation, for example by a metered inhaler. Dosage forms for topical administration include cremes, gels, ointments, salves, patches and transdermal delivery systems.

Pharmaceutically acceptable salts of compounds that can be used in the present invention can be formed with various organic and inorganic acids and bases. Exemplary acid addition salts comprise acetate, adipate, alginate, ascorbate, benzoate, benzenesulfonate, hydrogensulfate, borate, butyrate, citrate, caphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, nitrate, oxalate, pectinate, persulfate, 3-phenylsulfonate, phosphate, picate, pivalate, propionate, salicylate, sulfate, sulfonate, tartrate, thiocyanate, toluenesulfonate, such as tosylate, undecanoate and the like. Exemplary base addition salts comprise ammonium salts, alkali metall salts, such as sodium, lithium and potassium salts; earth alkali metall salts, such as calcium and magnesium salts; salts with organic bases (such as organic amines), such as benzazethine, dicyclohexylamine, hydrabine, N-methyl-D-glucamine, N-methyl-D-glucamide, t-butylamine, salts with amino acids, such as arginine, lysine and the like.

Pharmaceutically acceptable solvates of compounds that can be used in the present invention may exist in the form of solvates with water, for example hydrates, or with organic solvents such as methanol, ethanol or acetonitrile, i.e. as a methanolate, ethanolate or acetonitrilate, respectively.

Pharmaceutically acceptable prodrugs of compounds that can be used in the present invention are derivatives which have chemically or metabolically cleavable groups and become, by solvolysis or under physiological conditions, the compounds of the invention which are pharmaceutically active in vivo. Prodrugs of compounds that can be used in the present invention may be formed in a conventional manner with a functional group of the compounds such as with an amino or hydroxy group. The prodrug derivative form often offers advantages of solubility, tissue compatibility or delayed release in a mammalian organism (see, Bundgaard, H., Design of Prodrugs, pp. 7-9, 21-24, Elsevier, Amsterdam 1985).

These pharmaceutical compositions described herein can be administered to the subject at a suitable dose. The dosage regiment will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. Generally, the regimen as a regular administration of the pharmaceutical composition should be in the range of 0.1 μg to 5000 mg units per day, in some embodiments 0.1 μg to 1000 mg units per day. If the regimen is a continuous infusion, it may also be in the range of 0.1 ng to 10 μg units per kilogram of body weight per minute, respectively. Progress can be monitored by periodic assessment.

The present invention also provides for a method of treatment, amelioration or prevention of disorders or diseases which are due to (or which are linked to) biochemical and/or biophysical processes which take place in, on or within lipid raft structures of a mammalian cell. Corresponding diseases/disorders are provided herein above and corresponding useful compounds to be administered to a patient in need of such an amelioration, treatment and/or prevention are also disclosed above and characterized in the appended examples and claims. In a most preferred setting, the compounds (disrafters) described herein are used in these treatment methods by administration of said compounds to a subject in need of such treatment, in particular a human subject.

Due to the medical importance of the disrafting compounds described in context of the present invention, the invention also provides for a method for the preparation of a pharmaceutical composition which comprises the admixture of the herein defined compound with one or more pharmaceutically acceptable excipients. Corresponding excipients are mentioned herein above and comprise, but are not limited to cyclodextrins. As pointed out above, should the pharmaceutical composition of the invention be administered by injection or infusion it is preferred that the pharmaceutical composition is an emulsion.

The following examples illustrate this invention.

### EXAMPLE 1

### Synthesis of Compound 10ad: cis-Δ¹⁷⁽²⁰⁾-5alpha-Pregnen-3beta-ol

A suspension of sodium hydride (2.4 g, 59.8 mmol, 60% dispersion in mineral oil) in anhydrous dimethylsulfoxide (50 mL) was stirred at 70-75° C. for about 45 min under an atmosphere of argon. The resulting pale greenish solution was cooled to room temperature and a solution of commercially available ethyltriphenylphosphonium iodide in anhydrous dimethylsulfoxide (100 mL) was added. The obtained red solution was allowed to stand for about 5 to 10 min, then a solution of commercially available epiandrosterone (4 g, 13.79 mmol) in anhydrous dimethylsulfoxide (100 mL) was added and the resulting red reaction mixture was stirred at 55-60° C. for about 18 h under an argon atmosphere.

After cooling to room temperature, the reaction mixture was poured into ice/water (about 1 L) followed by extraction with diethyl ether (3×800 mL). The combined organic layers were washed repeatedly with water (4×1 L) to remove remaining dimethylsulfoxide, dried over sodium sulfate and the solvent was removed under reduced pressure. The crude product was subjected to purification by column chromatography on silica (petroleum/ethyl acetate 4:1) to provide 3.51 g (84%) of 10ad as a colourless solid.

¹H-NMR (300 MHz, CDCl₃): delta=0.63-0.73 (m, 2H), 0.82 (s, 3H), 0.87 (s, 3H), 0.90-1.83 (m, 20H), 2.10-2.26 (m, 2H), 2.31-2.40 (m, 1H), 3.54-3.63 (m, 1H), 3.65 (br s, 1H), 5.08-5.15 (m, 1 H).

(NMR shows the corresponding trans-isomer in less than 5%.)

MS (El): m/z=302 (M⁺).

### EXAMPLE 2

### Synthesis of Compound 10ae: cis-Δ¹⁷⁽²⁰⁾-5alpha-Pregnen-3-one

A solution of pyridinium chlorochromate (456 mg, 2.11 mmol) in dichloromethane (3 mL) was added to a solution of cis-Δ¹⁷⁽²⁰⁾-5alpha-pregnen-3beta-ol (10ad) in dichloromethane (4 mL). The resulting reaction mixture was stirred for about 18 h at room temperature and then filtered through a short column of silica using dichloromethane as eluent. After removal of the solvent under reduced pressure the product was obtained as colourless solid (210 mg, 83%).

¹H-NMR (300 MHz, CDCl₃): delta=0.73-0.82 (m, 2H), 0.89 (s, 3H), 0.92-0.98 (m, 1H), 1.02 (s, 3H), 1.10-1.62 (m, 10H), 1.63-1.67 (m, 3H), 1.71-1.79 (m, 1H), 1.99-2.45 (m, 8H), 5.08-5.16 (m, 1H).

(NMR shows the corresponding trans-isomer in less than 5%.)

MS (El): m/z=300 (M⁺).

### EXAMPLE 3

### Synthesis of Compound 10ai: 5alpha-Androstan-3alpha-ol

A mixture of commercially available androsterone (1.2 g, 4.14 mmol) and toluenesulfonylhydrazid (1.08 g, 5.79 mmol) in methanol (70 mL) was stirred at reflux temperature for 18 h. After cooling to room temperature, solid sodium borohydride (3.46 g, 91 mmol) was added in portions over a period of 1 h. The resulting reaction mixture was stirred at reflux temperature for 16 h. After removal of the solvent under reduced pressure, the residue was dissolved in dichloromethane (600 mL) and washed subsequently with water (1 L), dilute aqueous sodium carbonate (1 L), 1M aqueous hydrochloric acid (1 L), and again water (1 L). The organic layer was dried over sodium sulfate and the solvent was removed under reduced pressure to afford a colorless solid (1.53 g). Unexpectedly, analytical evaluation of that material showed the toluenesulfonyl hydrazone of androsterone. The material was dissolved in THF (25 mL) followed by addition of sodium borohydride (1.38 g, 36.4 mmol). The resulting reaction mixture was stirred at reflux temperature for 20 h. The solvent was removed under reduced pressure, and the residue dissolved in diethyl ether (500 mL), washed subsequently with water (500 mL), saturated aqueous sodium carbonate solution (500 mL), 1M hydrochloric acid (500 mL) and water (500 mL). After drying of the organic layer over sodium sulfate, the solvent was removed under reduced pressure and the crude material was subjected to purification by column chromatography (dichloromethane/ethyl acetate 4:1 to 2:1). Compound 10ai was obtained as a colorless solid (80 mg, 7%).

¹H-NMR (300 MHz, CDCl₃): delta=0.69 (2, 3H), 0.75-1.03 (m, 4H), 0.78 (s, 3H), 1.11-1.47 (m, 10H), 1.51-1.72 (m,11H),4.04 (m, 1H).

MS (El): m/z=276 (M⁺).

### EXAMPLE 4

### Synthesis of Compound 10ac: 3alpha-Methoxy-5alpha-androstane

A solution of 10ai (72 mg, 0.26 mmol) in DMF (3 mL) was added to solid sodium hydride (15 mg, 0.31 mmol, 60% dispersion in mineral oil) under an atmosphere of argon, and the resulting suspension was stirred for 10 min at room temperature. Neat methyl iodide (74 mg, 0.52 mmol) was added and the reaction mixture was stirred for 18 h at room temperature. The mixture was poured in water (500 mL) and extracted with diethyl ether (400 mL). The organic layer was washed with water (2×500 mL), dried over sodium sulfate, and the solvent was removed under reduced pressure. The crude material was purified by column chromatography on silica (petroleum/ethyl acetate 10:1) to afford 10ac as a colorless oil (32 mg, 42%).

¹H-NMR (300 MHz, CDCl₃): delta=0.68 (s, 3H), 0.75-1.10 (m, 4H), 0.79 (s, 3H), 1.12-1.32 (m, 8H), 1.35-1.72 (m, 11H), 1.78-1.83 (mn, 1H), 3.29 (s, 3H), 3.43 (m,1H).

### EXAMPLE 5

### Synthesis of Compound 10af: 5alpha-Pregnan-3beta-ol

A solution of compound 10ad (510 mg, 1.69 mmol) and palladium on charcoal (360 mg, 0.34 mmol, 10% palladium) in a mixture of ethanol (4 mL) and dichloromethane (4 mL) was stirred for 24 h at room temperature under an atmosphere of hydrogen. The reaction mixture was filtered through a pad of celite using dichloromethane as eluent. The solvent was removed under reduced pressure to afford analytically pure 10af as a colorless solid (514 mg, 100%).

¹H-NMR (300 MHz, CDCl₃): delta=0.55 (s, 3H), 0.78-1.84 (m, 26H), 0.81 (s, 3H), 0.86 (t, J=7.2 Hz, 3H), 3.59 (m, 1H).

MS (ESl): m/z=304 (M⁺).

### EXAMPLE 6

### Synthesis of compound 10ag: 5alpha-Pregnan-3alpha-azide

Compound 10af was transformed to the corresponding mesylate in an analogous manner as described for 10ad below.

A solution of that mesylate (240 mg, 0.63 mmol) and sodium azide (407 mg, 6.27 mmol) in dimethylsulfoxide (15 mL) was stirred at 90° C. for 20 h under an atmosphere of argon. The reaction mixture was poured in water (500 mL), extracted with dichloromethane (400 mL), and the combined organic layers were washed with water (3×500 mL). After drying over sodium sulfate, the solvent was removed under reduced pressure and the crude product was purified by column chromatography on silica (petroleum). Compound 10ag was obtained as a colorless oil (140 mg, 68%).

¹H-NMR (300 MHz, CDCl₃): delta=0.75 (t, J=7.4 Hz, 3H), 0.77 (s, 3H), 0.85-1.13 (m, 4H), 0.94 (s, 3H), 1.17-1.32 (m, 4H), 1.39-2.04 (m, 16H), 2.17-2.20 (m, 1H), 3.90 (m, 1H).

IR (neat): v=2099.74, 2083.14 cm⁻¹ (N₃).

### EXAMPLE 7

### Synthesis of Compound 10ah: cis-Δ¹⁷⁽²⁰⁾-5alpha-Pregnen-3alpha-azide

A solution of 10ad (1.13 g, 3.74 mmol) and 4-(dimethylamino)pyridine (548 mg, 4.49 mmol) in dichloromethane (30 mL) was cooled to 0° C. and neat mesyl chloride (473 mg, 4.12 mmol) was added. The resulting reaction mixture was allowed to warm to room temperature and stirred overnight. After 18 h the mixture was poured in water (1 L) and extracted with ethyl acetate (800 mL). The organic layer was washed with water (2×1 L), dried over sodium sulfate, and the solvent was removed under reduced pressure. The crude product was purified by column chromatography on silica (petroleum/ethyl acetate 4:1) to provide the corresponding mesylate as a colorless solid (1.3 g, 91%).

A solution of the mesylate (890 mg, 2.34 mmol) and sodium azide (1.21 g, 18.69 mmol) in N,N′-dimethyl-N,N′-trimethyleneurea (DMPU) (10 mL) was stirred at 60° C. for 2 days under an atmosphere of argon. The reaction mixture was poured in water (500 mL), extracted with dichloromethane (2×400 mL), and the combined organic layers were washed with water (2×1 L). After drying over sodium sulfate, the solvent was removed under reduced pressure and the crude product was purified by column chromatography on silica (petroleum). Compound 10ah was obtained as a colorless solid (445 mg, 58%).

¹H-NMR (300 MHz, CDCl₃): delta=0.75-0.99 (m, 2H), 0.80 (s, 3H), 0.86 (s, 3H), 1.01-1.29 (m, 5H), 1.32-1.72 (m, 15H), 2.17-2.37 (m, 3H), 3.89 (m, 1H), 510 (m, 1H).

IR (neat): v=2105.38, 2081.46 cm⁻¹ (N₃).

### EXAMPLE 8

### Synthesis of Compound 10aj: cis-Δ¹⁷⁽²⁰⁾-Dodecylidene-3beta-androstanol

Freshly prepared dodecylidene ylide (50.7 mmol) was added to a solution of commercially available epiandrosterone (4.21 g, 14.5 mmol) in dry dimethylsulfoxide (160 mL) and the mixture was stirred at 70° C. for 24 h. The ylide was prepared from commercially available dodecyltriphenylphosphonium bromide and sodium hydride in dry dimethylsulfoxide in an analogous manner as described for compound 10ad. After quenching with water (400 mL) and extraction with diethyl ether (6×200 mL), the combined organic layers were dried over sodium sulfate and the solvent was removed under reduced pressure. Purification of the crude product by column chromatography on silica (petroleum/ethyl acetate 5:1) provided compound 10aj as a colorless solid (2.17 g, 34%).

¹H-NMR (300 MHz, CDCl₃): delta=0.74 (m, 8H), 1.05 (m, 27H), 1.28 (m, 10H), 1.98 (m, 6H), 3.49 (m,1H), 4.92 (m,1H).

MS (ESl): m/z=443 ([M+H]⁺).

### EXAMPLE 9

### Synthesis of Compound 10ak: cis-Δ¹⁷⁽²⁰⁾-Dodecylidene-3-androstanone

Pyridinium chlorochromate (120 mg, 0.55 mmol) was added to a solution of compound 10aj (4.21 g, 14.5 mmol) in dichloromethane (5 mL) and the resulting mixture was stirred for 3 h at room temperature. Purification of the crude reaction mixture by column chromatography on silica (dichloromethane/ethyl acetate mixtures) provided compound 10ak as a colorless solid (119 mg, 99%).

¹H-NMR (300 MHz, CDCl₃): delta=0.75-1.03 (m, 9H), 1.10-1.76 (m, 32H), 1.98-2.39 (m, 10H), 5.01 (m, 1H).

MS (ESl): m/z=441 ([M+H]⁺).

### EXAMPLE 10

### Synthesis of Compound 10al: cis-Δ¹⁷⁽²⁰⁾-Dodecylidene-3alpha-aminoandrostan

Neat mesylchloride (456 mg, 3.98 mmol) was added to a solution of compound 10aj (1.6 g, 3.61 mmol) and 4-(dimethylamino)pyridine (525 mg, 4.3 mmol) in dichloromethane (20 mL) at 0° C. The resulting reaction mixture was gradually warmed to room temperature and stirred for 60 h. After quenching with water (100 mL), the mixture was extracted with ethyl acetate (3×200 mL) and the combined organic layers were dried over sodium sulfate. The solvent was removed under reduced pressure and the obtained crude product was used in the next transformation. The mesylate was obtained as a colorless solid (1.9 g, 99%). Sodium azide (4.1 g, 15.4 mmol) was added to a solution of that mesylate (1.9 g, 3.65 mmol) in dimethylformamide (15 mL) and the reaction mixture was stirred at 105° C. for 16 h under an atmosphere of argon. The solvent was removed under reduced pressure, the residue was dissolved in ethyl acetate (100 mL) and washed with water (2×100 mL). The combined organic layers were dried over sodium sulfate and the solvent was removed under reduced pressure. Purification of the crude product by column chromatography on silica (petroleum/ethyl acetate 100:1) provided the corresponding azide as a colorless solid (1.14 g, 67%). The material was directly submitted to the following transformation.

A solution of that azide (470 mg, 1 mmol) in dry diethyl ether (10 mL) was added to a solution of lithium aluminum hydride (190 mg, 5 mmol) in dry diethyl ether (10 mL) at reflux temperature. The resulting reaction mixture was stirred at reflux temperature for further 18 h, then cooled to room temperature and diluted with methanol (200 mL). After quenching with water (500 mL), the mixture was extracted with dichloromethane (2×250 mL) and the combined organic layers were dried over sodium sulfate. The solvent was removed under reduced pressure and the crude material purified by column chromatography on silica (petroleum/dichloromethane 3:2). Compound 10al was obtained as a colorless solid (175 mg, 40%).

¹H-NMR (300 MHz, CDCl₃): delta=0.66 (m, 3H), 0.79 (m, 6H), 1.15 (m, 27H), 1.41 (m, 6H), 1.62 (m, 9H), 3.12 (br s, 1H), 5.23 (m, 1H).

### EXAMPLE 11

### Synthesis of Compound 10da: cis-Δ¹⁷⁽²⁰⁾-19-Norpregna-1,3,5(10),17(20)-tetraen-3-ol

A suspension of sodium hydride (680 mg, 16.7 mmol, 60% dispersion in mineral oil) in dry dimethylsulfoxide (20 mL) was stirred for 90 min at 72° C. under an atmosphere of argon. After cooling to room temperature, a solution of commercially available ethyltriphenylphosphonium iodide (7 g, 16.7 mmol) in dry dimethylsulfoxide (25 mL) was added and the resulting red solution was stirred for about 15 min at room temperature. A solution of commercially available estrone (1 g, 3.7 mmol) in dry dimethylsulfoxide (12 mL) was added and the reaction mixture was stirred for 18 h at 60° C. After cooling to room temperature, water (20 mL) was added and the resulting yellow mixture was poured into water (1 L) and extracted with diethyl ether (1 L). The organic layer was washed thoroughly with water (4×1 L), dried over sodium sulfate, and the solvent was removed under reduced pressure to afford the crude product. Purification was achieved by column chromatography on silica (dichloromethane) and 10da was obtained as a colorless solid (976 mg, 94%).

¹H-NMR (300 MHz, CDCl₃): delta=0.91 (s, 3H), 1.27-1.60 (m, 5H), 1.68-1.79 (m, 5H), 1.89-1.94 (m, 1H), 2.21-2.47 (m, 5H), 2.82-2.86 (m, 2H), 4.59 (br s, 1H), 5.12-5.19 (m, 1H), 6.56 (d, J=2.7 Hz, 1H), 6.63 (dd, J=8.4, 2.7 Hz, 1H), 7.16 (d, J=8.4Hz, 1H).

MS (El): m/z=282 (M⁺).

### EXAMPLE 12

### Synthesis of Compound 10db: cis-Δ¹⁷⁽²⁰⁾-19-Norpregna-1,3,5(10),17(20)-tetraen-3-yl acetate

Neat acetic anhydride (81 mg, 0.79 mmol) was added to a solution of compound 10da (160 mg, 0.57 mmol) and 4-(dimethylamino)pyridine (97 mg, 0.79 mmol) in dichloromethane (4 mL), and the resulting reaction mixture was stirred at room temperature for 18 h. The reaction mixture was poured into water (500 mL) and extracted with ethyl acetate (500 mL). The organic layer was washed with water (500 mL), dried over sodium sulfate, and the solvent was removed under reduced pressure to afford the crude product. Purification by column chromatography on silica (dichloromethane) provided compound 10db as a colorless solid (151 mg, 82%).

¹H-NMR (300 MHz, CDCl₃): delta=0.91 (s, 3H), 1.28-1.65 (m, 5H), 1.66-1.74 (m, 5H), 1.79-1.95 (m, 1H), 2.12 (s, 3H), 2.23-2.48 (m, 5H), 2.87-2.98 (m, 2H), 5.12-5.19 (m, 1H), 6.63 (d, J=2.8 Hz, 1H), 6.71 (dd, J=8.5, 2.8 Hz, 1H), 7.21 (d, J=8.5 Hz, 1H).

MS (El): m/z=324 (M⁺).

### EXAMPLE 13

### Synthesis of Compound 10dc: cis-Δ¹⁷⁽²⁰⁾-19-Norpregna-1,3,5(10),17(20)-tetraen-3-yl methyl ether

Neat methyl iodide (106 mg, 0.74 mmol) was added to a suspension of 10da (105 mg, 0.37 mmol) and sodium hydride (23 mg, 0.56 mmol, 60% dispersion in mineral oil) in dry dimethylformamide (6 mL) under an atmosphere of argon and the resulting reaction mixture was stirred at room temperature for 18 h. The reaction mixture was poured in water (1 L) and extracted with ethyl acetate (700 mL). The organic layer was washed thoroughly with water (3×800 mL), dried over sodium sulfate, and the solvent was removed under reduced pressure. The crude product was purified by column chromatography on silica (dichloromethane). Compound 10dc was obtained as a colorless solid (36 mg, 33%).

¹H-NMR (300 MHz, CDCl₃): delta=0.91 (s, 3H), 1.26-1.62 (m, 5H), 1.68-1.74 (m, 5H), 1.79-1.95 (m, 1H), 2.21-2.47 (m, 5H), 2.85-2.95 (m, 2H), 3.78 (s, 3H), 5.12-5.19 (m, 1H), 6.63 (d, J=2.7 Hz, 1H), 6.71 (dd, J=8.6, 2.7 Hz, 1H), 7.21 (d,J=8.6 Hz, 1H).

MS (ESl): m/z=296 (M⁺).

### EXAMPLE 14

### Disrafter Assay, Disrafter-Liposome Raftophile Assay (D-LRA)

In accordance with the present invention, the disrafting capacity of a given compound and its medical usefulness in the amelioration, treatment or prevention of a disease related to lipid raft processes may be tested by a D-LRA provided herein.

The raftophilicity of certain fluorescent indicators varies with the raft content of liposomes which, in turn, is determined by their lipid composition and the presence of raft modulators.

The D-LRA assay detects two extremes of raft modulation, disrafting and raft augmentation. % disrafting below 0 results from an actual increase in partition of the indicator, caused by an increased raft content of the liposomes. This can result from a restructuring of the rafts, i.e. an increased density, or physical insertion of the test compounds into the liposomes increasing raft quantity. Significance can be ascribed to values above 25% (disrafting) and below −25% (disrafters by “augmentation”).

Liposomes (defined below) with a raft content of about 50% are incubated with potential disrafters. The change in raft content is then determined with an indicator (standard raftophile).

### Material for D-LRA

1. Liposomes

Raft liposomes: (35% cholesterol, 10.5% sphingomyelin (SM), 3.5% GM1, 25.5% phosphatidylethanolamine (PE) and 25,5% phosphatidylcholine (PC)) Non-raft liposomes: N liposomes (50% PE, PC)

Liposomes are prepared by spreading lipids dissolved in tert. butanol on a glass surface at 50 ° C. in a rotary evaporator rinsed with nitrogen. After 6 h desiccation the lipids are taken up in 40 mM octyl-β-D-glucoside (OG) to a concentration of 1 mg/ml and dialysed for 24 h against 2 changes of 5 l PBS with 25 g Biobeads (Amberlite XAD-2) at 22° C.
2. Indicators

Indicators are fluorescent compounds which preferentially partition into rafts. These are selected to represent different structural classes, and different excitation/emission wavelengths. This is important when raft modulators are tested which interfere with indicator fluorescence. 2.1. Perylene is a raftophilic compound which embeds completely into membranes.

2.2. GS-96 is a raftophilic adduct of the general structure cholesterol-linker-rhodamine-peptide (only the cholesterol is membrane-inserted). The structure of GS-96 is Cholesteryl-Glc-RR-βA-D(Rho)-βA-GDVN-Sta-VAEF (one-letter amino acid code; Glc=glycolic acid, βA=β-alanine, Rho=rhodamine, Sta=statine; Fmoc-Statine Neosystem FA08901, Strasbourg, France) and was generated by applicant using standard procedures: peptide synthesis was carried out on solid support using the 9-fluorenylmethyloxycarbonyl (Fmoc) method with piperidine as deprotecting reagent and 2-(1H-benzotriazol-1-yl)-1,1,3,3-tetramethyluronium hexafluorophosphate (HBTU) as coupling reagent employing an Applied Biosystems 433A peptide synthesizer. Fmoc-protected amino acid building blocks are commercially available, except of rhodamine-labelled Fmoc-glutamic acid, which was prepared by a modified procedure extracted from literature (T. Nguyen, M. B. Francis, Org. Lett. 2003, 5, 3245-3248) using commercially available Fmoc-glutamic acid tert-butyl ester as substrate. Final saponification generated the free acid used in peptide synthesis. Cholesteryl glycolic acid was prepared as described in literature (S. L. Hussey, E. He, B. R. Peterson, Org. Lett. 2002, 4, 415-418) and coupled manually to the amino function of the N-terminal arginine. Final cleavage from solid support using standard procedures known in peptide synthesis and subsequent purification by preparative HPLC afforded GS-96.

2.3. J-12S is a smaller adduct serving the same purpose: Cholesteryl-Glc-RR-βA-D(Rho). Other indicators, e.g. sphingomyelin adducts, are equally suitable.

### Sketched Method of D-LRA

Liposomes are diluted into PBS to a final lipid concentration of 200 μg/ml (R: 302 μM, N: 257 μM total lipid)
Preincubate 100 μl liposomes 30 min 37 ° C. on a thermomixer (1000 rpm)
Add 1 μl test compound stock solution (100 μM final concentration) or appropriate solvent controls and incubate 2 h as above
Add indicator (GS-96 0.2 μM or perylene 2 μM) and incubate a further 1 h
Proceed as for LRA: centrifuge 20 min in the TLA-100 rotor of the Beckman Optima centrifuge at 400 000 g and 37° C.
Withdraw the top 50 μl of the supernatant (S) and transfer to a microtiter plate containing 150 μl 50.3 mM OG
From tubes incubated in parallel transfer the total liposomes (L) to microtiter wells containing 100 μl 80 mM OG
Wash the tubes with 200 μl 40 mM OG (GS-96) or 100 mM C8E12 (perylene) at 50 ° C. on the thermomixer (1400 rpm) to elute adherent (A) indicator and transfer content to microtiter plate
Prepare 200 p, indicator concentration standards in 40 mM in the microtiter plate
Determine the indicator concentrations in S, L and A in a fluorimeter/plate reader (Tecan Safire)
Compute partition coefficients CpN, CpR and raftophilicity (rΦ=CpR/CpN) with respect to CpN
Calculate disrafting activity as % disrafting=100 (rΦ_{control}−rΦ_{test compound})/rΦ_{control}

### Detailed Method

N and R Liposomes were diluted into PBS to a final lipid concentration of 200 μg/ml and 100 μl aliquots preincubated 30 min 37 ° C. on a thermomixer (1000 rpm).

1 μl of DMSO (solvent controls) and the test compound stock solutions (all 10 mM in DMSO, except where noted) were added and incubated 2 h as above.

1 μl, indicator in DMSO was then added (final indicator concentrations GS-96 0.2 μM, perylene 2 μM) and incubation continued for 1 h as above

Incubation mixes were centrifuged 20 min in the TLA-100 rotor of the Beckman Optima centrifuge at 400 000 g (37 ° C.). 50 μl of the supernatant (S) was transferred from the top of the tube to a 96-well microtiter plate containing 150 μl 53.3 mM OG in PBS.

From tubes incubated in parallel the total liposomes (L) were transferred to microtiter wells containing 100 μl 80 mM OG in PBS. The tubes were then washed with 200 μl 40 mM OG (GS-96) or 100 mM C8E12 (perylene) at 50 ° C. on the thermomixer (1400 rpm) to elute adherent (A) indicator and content transferred to the microtiter plate.

200 μl indicator concentration standards were prepared in 40 mM OG in the microtiter plate.

The 96-well plate was read in a fluorimeter/plate reader (Tecan Safire) at the appropriate wavelengths, excitation 411 nm, emission 442 nm (perylene); excitation 553 nm, emission 592 nm (GS-96). Based on the concentration standards fluorescence readings were converted to indicator concentrations.

From the concentration data partition coefficients CpN and CpR were computed as follows:
The indicator concentrations in the respective phases are denoted L (in total liposomes), A (adherent to the tube wall), S (in the aqueous phase).
Cp=f*(L-S)/S. f*(L-S) is the compound concentration in the membrane, where f is the ratio of incubation volume to actual lipid bilayer volume.
The raftophilicity was calculated as the ration of the two partition coefficients, rΦ=CpR/CpN.

Disrafting activity was calculated as follows:

% disrafting=100*(rΦ_{control}−rΦ_{test compound})/rΦ_{control}.

Results: Androsterone, epiandrosterone and cholesterol gave in this test 0% and are, accordingly, no disrafters in accordance with the present invention. Yet, compounds 10ac, 10ad, 10ae, 10af, 10ag, 10ak, 10al, 10da, 10db and 10dc provided in the DLRA assay high negative or positive values, respectively, and can be considered as disrafters in context of the present invention which may be employed as corresponding pharmaceuticals. In particular, 10ad provided in the DLRA with perylene a value of about −105% and with GS-96 a value of about −66%, 10ae provided for corresponding values of about −43% (with perylene) and −20% (with GS-96), evaluation of 10af resulted in −217% (with perylene) and −74.7% (with GS-96), 10ag provided for −71.8% (with perylene) and −187% (with GS-96). Similarly, 10al afforded −216% (with perylene), 10da −729% (with perylene), 10db −536% (with perylene) and 10dc −139% (with perylene). Thus, these compounds are capable of increasing the size of lipid rafts by augmentation and are. considered disrafters in the context of this invention. In contrast, compound 10ac, when tested in the same experimental setting, provided in the DLRA with perylene a value of about +68%. Therefore, compound 10ac is able to exert raft modulation by disrafting according the above given definition and is also considered a disrafter in the context of the present invention.

### EXAMPLE 15

### Virus Budding Assay (Influenza Assay)

The aim of this assay is the identification of compounds targeting raft-dependent virus budding and to distinguish from inhibitor effects on other stages of virus reproduction.

### Principle of Virus Budding Assay

Nascent virus (influenza) on the cell surface is pulse-biotinylated 6 or 13 h post infection and treated with test compounds for 1 h. Biotinylated virus is captured on a streptavidin-coated microtiter plate. Captured virus is detected with virus-specific primary and peroxidase-labeled secondary antibody. A luminescent signal generated from a peroxidase substrate is recorded with a CCD camera (LAS 3000). Intensities are evaluated by densitometry.

Value less than 100% reveal inhibition of virus budding. Significance can be ascribed to values below 80%, preferably below 70%. Values above 100% mean that more viruses are released than in the untreated control. This reflects a change in regulation of virus release which can have various causes. In this case significance can be ascribed to values above 130%. These will be followed up if the compound is inhibitory in an assay of virus replication.

### Materials of Virus Budding Assay

1. Infection
96-well plate MDCK 1-2 d
Influenza virus stocks

IM (infection medium): MEM+Earle's (Gibco/InVitrogen 21090-022) plus 2 mM L-glutamin, 10 mM Hepes, bovine serum albumin (BSA) 0.2%
2. Biotin labelling
stock solutions: 20% glucose (about 1 M),1 M glycin
PBS8G: PBS pH 8, 1 mM glucose, ice-cold
biotin, 20 μg -100 μl—per well of 96-well plate, 1 mg biotin/5 ml PBS8G freshly prepared on ice
Quench medium (IM, 10 mM glycine), ice cold
3. Chase and harvest
Aluminum thermoblocks for plate T shift and test compound dilutions
IM±test compounds, 37° C.
TBS (Tris-buffered saline pH 7.4, 10 mM Tris, 150 mM NaCl); TBS⁺⁺⁺=TBS plus protease inhibitors: dilute 5% trypsin inhibitor 1:250, 200 mM AEBSF 1:200 and 1 mg/ml aprotinin 1:100.
ice-cold 96-well plates (v-bottom) and MP3300 multiwell plate rotor of the Multifuge 1-S—R (Heraeus) centrifuge 2 ° C.
4. Capture
streptavidin-coated 96-well plate Reacti-Bind™ Streptavidin HBC (Pierce 15500)

### Sketched Method of Virus Budding Assay

1. Infection and neuraminidase treatment wash wells with 2×200 μl IM. Infect with 100 μl virus diluted in IM at a multiplicity of infection 0.5-2 infectious units per cell for 30 min at 37° C. Remove incoculum and replace by 150 μl IM.
incubate for 6 or 13 h post-infection (p.i.)
2. Biotinylation
place plate on ice, wash 4×0.20 ml ice-cold PBS8G
add 0.1 biotinylation solution in PBS8G per well
rock 12 min on ice in refrigerator
wash 5× with 0.25 ml quench medium on ice
3. Budding/chase
transfer plate to preheated aluminum block
exchange last wash for 125 μl pre-warmed medium ± test compounds (i.e. compounds to be tested and considered as “disrafters”, “disrafting compounds in D-LRA described above)
return plate on block to incubator for 1 h 37° C.
4. Harvest
place on ice
transfer 50 μl overlays to v-bottom centrifugation plate containing 50 μl TBS+++ on ice (1:1 dilution)
centrifuge the plate 30 min 2° C. 4400 rpm
alternative equivalent protocol: transfer overlays to Millipore (MSDVS6510) clear filtration plates MS HTS™ DV, 0.65 μm hydrophilic low protein binding and centrifuge 1 min, 1500g, into a Nunc assay plate.
5. Capture
prepare streptavidin-coated plate by washing with 3×200 μl TBS/0.1% Tween and once with TBS
transfer 50 μl virus overlay supernatants to capture plate
capture on rocker 2 h at 37° C. or over-night at 4° C.
6. Detection
to capture plate add 50 μl TBS, 40 mM OG and incubate on a rocker for 20 min at 4 ° C.
wash 1× with 200 μl TBS add 200 μl block and incubate 2 h at room temperature or over-night at 4 ° C.
develop with antiNP monoclonal (MAb pool 5, US Biological l7650-04A) diluted 1:1000 in block, 1 h at room temperature and wash 3×
use rabbit anti-mouse-peroxidase conjugate 1:2000 as secondary antibody, 1 h at room temperature and wash 3×
develop with Pierce Super Signal (West-Dura) luminescent, or fluorescent or colorimetric substrate
image with CCD camera (LAS 3000, Raytest) and quantify densitometrically

Results: It is exemplified that virus budding was reduced by using 10ad to 61%, whereas 10ae provided for enhanced virus budding of about 140%. Percentage values are given with respect to an untreated control. These compounds are therefore suitable compounds for the development of pharmaceutical compositions used for the treatment of influenza infection. Nevertheless, effects observed in the influenza virus reproduction and infectivity assay (cf. the following example) are further experimental results to be used to demonstrate the usefulness of the compounds provided in the present invention in a medical setting.

### EXAMPLE 16

### Virus Reproduction and Infectivity Assay (Focus Reduction Assay)

The aim of this assay is identifying disrafting compounds inhibiting virus replication or lowering virus infectivity.

### Principle

Assay of antiviral effects under conditions of virus titration, equivalent to a traditional plaque reduction assay, except that it is done on microtiter plates and developed as a cell Elisa. Cells are briefly preincubated with test compound dilutions and then infected with serially diluted virus.

### Materials

Low retention tubes and glass dilution plate ((Zinsser) from 70% EtOH, dried under hood)
2 Thermomixers, 1.5 ml Eppendorf and 96-well blocks
96-well plates MDCK cells 1-2 d
Virus aliquots with known titer
IM (infection medium)
trypsin 1 or 2 mg/ml stock solution, freshly prepared.
glutaraldehyde (Sigma, ampoules, kept at −20° C.)
0.05% in PBS (dilute 1:500), freshly prepared, 250 ml per plate
Antibodies for cell Elisa development; Pierce SuperSignal (West Dura) substrate

### Method

1. Compound dilutions
Thaw out test compounds at 37° C. and sonicate if necessary
Preheat IM in low retention tubes at 37° C. in a thermomixer and add test compounds [μl] as follows:
100 μM: 1078 +22 μl
50 μM: 1089 +11 μl
25 μM: 1094.5 +5.5 μl
10 μM: 1098 +2.2
After at least 30 min shaking compound dilutions are transferred into a glass 96-well plate preheated in a thermomixer microplate block at 37° C.
For two titration plates one glass plate is sufficient, the left half receives the test media for plate 1, the right half for plate 2. Each well receives 250 μl test medium (see template below)
2. Infection
Predilute virus 1:64 in IM (630 μl+10 μl). Dilute virus in cold IM 1:2000 (=1) and then make 2 further two-fold dilutions. For one 96-well plate prepare 3, 1.5, 1.5 ml, for two plates 6, 3, 3 ml and keep at 4° C.
Weigh out trypsin, prepare a solution 20 μg/ml and put through a 0.2 μm sterile syringe filter. Then dilute to 4 μg/ml in IM.
Shortly before infection add 1 vol. trypsin (4 μg/ml) to virus dilutions or to IM (for mock infection) and keep at 4° C. until infection.
Wash monolayers 2×200 μl IM.
With a multichannel pipette add 100 μl test compounds or solvent controls in IM, so that each column (2 to 11) contains one test compound dilution. (1 and 12 receive IM and can serve as additional controls if edge effects are minimal.)
With a multichannel pipette add 100 μl IM, 2 μg trypsin/ml to rows A and H (mock infection). Add virus dilutions to the other rows, changing tips every time. After each addition pipet up and down.
Incubate 16 h at 37° C.
Microscopy: Assess toxicity/cell morphology/precipitation in mock-infected wells.
Terminate infection by fixing and immersing/filling the whole plate with 250 ml 0.05% glutaraldehyde for at least 20 min RT.
3. Detection
Shake off the glutaraldehyde and rinse with PBS.
Permeabilize 30 min with 50 μl 0.1% TX-100 in PBS and rinse with PBS.
Block 1 h on a rocker at RT or over-night at 4 ° C. in TBS/Tween/10% FCS.
Develop with anti-NP (MAb pool 5) diluted 1:1000 in block, 1 h RT and wash 3× with TBS/Tween.
Add peroxidase conjugated secondary anti-mouse antibody at about 1:2000, 1 h on a rocker at RT and wash 2× TBS/Tween, once with TBS.
4. Imaging
develop with SuperSignal West Dura (Pierce 34076).
image with CCD camera LAS 3000 (Fuji/Raytest) at high resolution: use Fresnel lense.
quantify by densitometry using mock-infected controls as background.

### Quantification of Assay Results

The edge columns of a 96-well plate with MDCK cell monolayers are non-infected but treated with test compound and serve as background controls (well a) for densitometric evaluation (see below). Three further wells b, c and d are infected with virus dilutions, e.g. 1:512 000, 1:256 000 and 1:128 000, so that the 1:128 000 dilution will generate 50 to 100 foci. Suitable dilutions were determined by virus titration.

Foci of infected cells are developed immunohistochemically. Initially all wells are blocked for 1 h or over night on a rocker with 200 μL per well of a mixture of PBS+10% heat-inactivated fetal calf serum (block). This is followed by lh with 50 μL per well antibody to viral nucleoprotein (MAb pool 5, US Biological l7650-04A) 1:1000 diluted in block. Antibody is removed by three times 5 min washes with TBS (Tris-buffered saline)/Tween (0.1%) (TT). The next incubation is 1 h with 50 μL per well rabbit-anti-mouse-HRP (coupled to horseradish peroxidase) 1:2000 diluted in block. Finally, two washes as above and one with TBS.

The last wash is removed quantitatively and replaced by 50 μL per well substrate (Pierce 34076). The plate is exposed 5 to 10 min through the pre-focused Fresnel lense of the LAS 3000 CCD camera (high resolution mode).

Images are evaluated densitometrically. Initially the background is subtracted (well a, see above). The densitometric intensity is calculated as follows:

I=[0.25×i(well b)+0.5×i(well c)+i(well d)]/1.75

wherein i is defined as 10000 times the intensity per area measured for the relevant well b, c or d. This calculation corresponds to the classical plaque assay. The factors represent the weighting of the individual values.

Results are expressed as % inhibition defined as follows:

% inhibition=100−% control

wherein % control is calculated by multiplying a given I for test compound by 100 and dividing by I for the appropriate solvent control. If I is a control or solvent control, its value is set as 100%.

Results: Two compounds, 10ae and 10af, both tested positive in the above-mentioned DLRA and were identified as disrafters. When evaluating their inhibitory effect in the PR8 virus replication assay, both provided good results. 10ae inhibited virus replication by 32.9% at a concentration of 50 μM, while 10af inhibited the same process by 27.9% at 50 μM concentration. Thus, both substances are preferred compounds for the pharmaceutical intervention in influenza infection. Two further the compounds, which tested positive in the DLRA, i.e. compounds 10ad and 10al, provided for particular good results in the influenza virus replication assay and are thus even more preferred compounds to be used in the pharmaceutical compositions described herein for the treatment of influenza infection. In the case of compound 10ad, PR8 virus replication was inhibited by 59.6% at a concentration of 20 μM compared to solvent vehicle alone. When using compound 10al at a concentration of 12.5 μM the virus replication was inhibited by 54%, thus making compound 10al an even more preferred compound for the treatment of influenza infection.

### EXAMPLE 17

### Degranulation Assay

Mast cells are a widely used model system for hyperallergic reactions or asthma. On their surface they express high affinity receptors for IgE (FcεRI). Upon binding of antigen-specific IgE to the receptor cells become sensitive to antigen (allergen). When sensitized cells encounter multivalent antigen the clustering of IgE-FcεRI complexes initiates a cascade of cellular events that ultimately leads to degranulation, that is release of mediators of inflammation and cellular activation, such as cytokines, eicosanoids, histamine and enzymes. Several steps in this cascade are raft-dependent, such as antigen-triggered relocation of FcεRI to rafts, disruption of the signaling complex assembled around LAT and/or dislocation of phosphoinositides, Ca²⁺-influx (raft localization of plasma membrane calcium channels), membrane ruffling (cytoskeletal reorganizations involving Akt/WASP/FAK) and exocytosis. Therefore, the assay can be used as a screening method to identify raft-modulating compounds, in particular compounds useful in the medical management of asthma. Especially in conjunction with other assays for pre-selection of raft-modulating compounds the assay is a powerful tool to demonstrate the effectiveness of such compounds for intervention in biological processes.
1. Introduction

The assay measures release of β-hexosaminidase as a marker of release of various preformed pharmacological agents in response to clustering of the high affinity IgE receptor (FcεRI) by means of multivalent antigen-IgE complexes. Rat basophilic leukemia (RBL-2H3) cells, a commonly used model of mast cell degranulation, are sensitized with anti-DNP specific IgE and challenged with multivalent DNP-BSA. The release of β-hexosaminidase into the supernatant is measured by enzymatic conversion of the fluorogenic substrate 4-methylumbelliferyl-N-acetyl-ε-D-glucosaminide to N-acetyl-β-D-glucosamine and highly fluorescent methylumbelliferone and quantified by fluorescence detection in a Tecan Safire™ plate reader.
2. Materials

### Chemicals and Specialty Reagents

Surfact-Amps X-100 solution was obtained from Pierce, DNP-bovine albumin conjugate (DNP-BSA) and 4-methylumbelliferyl-N-acetyl-β-D-glucosaminide (MUG) were from Calbiochem, tri(ethylene glycol) monoethyl ether (TEGME) from Aldrich, DMSO Hybri-Max and human DNP-albumin from Sigma. Rat anti-DNP IgE monoclonal antibody was acquired from Biozol. All cell culture media, buffers and supplements were obtained from Invitrogen except fetal calf serum (FCS) which was from PAA Laboratories (Cölbe, Germany). Other reagents were of standard laboratory quality or better.

Other chemicals are standard laboratory grade or better if not specified otherwise.

### Buffers and Solutions

Phosphate buffered saline (PBS) and 1 M HEPES were provided by the in-house service facility. Tyrode's buffer (TyB) consisted of Minimum Essential Medium without Phenol Red (Invitrogen) supplemented with 2 mM GlutaMAX™-I Supplement (Invitrogen) and 10 mM HEPES. Lysis buffer consisted of 25 mM Tris-HCl, pH 7.5, 150 mM NaCl, 5 mM EDTA and 1% (w/v) Triton X-100. Human DNP-BSA was dissolved to 1 mg/ml in Millipore water. MUG substrate solution was 2.5 mM 4-methylumbelliferyl-N-acetyl-β-D-glucosaminide 0.05 M citrate, pH 4.5 and stop solution was 0.1 M NaHCO₃/0.1 M Na₂CO₃, pH 10.

### Cell Culture

RBL-2H3 cells obtained from the German Collection of Microorganisms and Cell Cultures (Braunschweig, Germany) were maintained in 70% Minimum Essential Medium with Earle's Salts/20% RPMI 1640/10% heat-inactivated fetal calf serum) supplemented with 2 mM GlutaMAX™-I in 5% CO₂ at 37° C. and routinely checked to be free of mycoplasma contamination. Cells grown in 175 cm² flasks were split with 0.05% Trypsin/EDTA and resuspended in 20 ml fresh medium. One hundred and 50 μl cell suspension were plated per well into 24 well cluster plates (Costar, Schiphol-Rijk, Netherlands) and cells were used one or two days after plating, respectively.
3. Measurement of β-hexosaminidase release

### Method

Two to 24 hours before incubation with test compounds the medium was removed and cells were sensitized with 0.4 μg/ml anti-DNP IgE in fresh medium. Following sensitization, cells were washed once with warm TyB and incubated for 60 min with test compound at a maximum of 100 μM or the highest non-toxic concentration (total vehicle concentration adjusted to 1%) or 1% vehicle in TyB at 37° C. DNP-HSA (0.1 μg/ml final concentration) or buffer alone was added and cells incubated for 15 min at 37° C. Plates were centrifuged at 4° C. for 5 min at 250×g and immediately transferred to ice. Supernatants were collected and the cells lysed with lysis buffer. Hexosaminidase activity in supernatants and lysates was measured by incubating 25 μl aliquots with 100 μl MUG substrate solution in a 96-well plate at 37° C. for 30 min. The reaction was terminated by addition of 150 μl stop solution. Fluorescence was measured in a Tecan Safire™ plate reader at 365 nm excitation and 440 nm emission settings.

### Quantification of Assay Results

Each compound is tested in duplicates in at least three independent experiments. β-hexosaminidase release is calculated after subtraction of unspecific release (release without addition of antigen) using the formula:

% degranulation=100×RFU supernatant/RFU lysate

Inhibition of 13-hexosaminidase release with respect to control is calculated as follows:

% inhibition=100×(1−(RFU supernatant of compound/RFU supernatant of control))

Values for CTB internalization from independent experiments are averaged and accepted when the standard deviation (SD)≦15%.

Results: One of the compounds, which tested positive in the DLRA, i.e. compound 10al, provided for a particular good result in the degranulation assay and is thus a preferred compound to be used in the pharmaceutical compositions described herein for the treatment of asthma and related immunological diseases. In the case of compound 10al the release of β-hexosaminidase was inhibited by 61% at a concentration of 100 μM compared to solvent vehicle alone.

### EXAMPLE 18

### Simian virus 40 (SV40) assay

Uptake of Simian Virus 40 (SV40) is a model for infection by diverse bacteria and viruses which utilize the raft domain to gain entry into the cell (Pelkmans (2002) Science 296, 535-539). In more detail, SV40 is transported to the endoplasmic reticulum upon caveolae-mediated endocytosis via caveosomes (Pelkmans (2001) Nature Cell Biol. 3, 473-483), as well as by non-caveolar, lipid raft-mediated endocytosis (Damm (2005) J. Cell Biol. 168, 477-488).

The SV40 assay described herein is used as a screen for compounds which may inhibit bacterial or viral infection at the stage of caveolar incorporation, endocytosis and early intracellular trafficking. This mechanism is particularly relevant to infection by respiratory syncytial virus, coronaviruses (e.g. causing SARS or upper respiratory tract infections) and Mycobacterium spp. leading to tuberculosis.

In contrast, vesicular stomatitis virus (VSV) enters cells via clathrin-mediated endocytosis into early and late endosomes (Sieczkarski (2003) Traffic 4, 333-343). Thus, the VSV assay described herein serves as a proof-of-concept counterscreen revealing compounds which gain entry into cells via a mechanism independent from caveolae/lipid raft-mediated endocytosis.

### Cell Culture

HeLa cells were obtained from DSMZ, Braunschweig, and maintained in D-MEM medium (Gibco BRL) without phenol red supplemented with 10% fetal bovine serum (FBS; PAN Biotech GmbH), 2 mM L-glutamine and 1% penicillin-streptomycin. The cells were incubated at 37° C. in 5% carbon dioxide. The cell number was determined with CASY cell counter (Schärfe System GmbH) and were seeded using the Multidrop 384 dispenser (Thermo). The following cell numbers were seeded per well (in 100 μL medium) in 96-well plates (Greiner) the day before adding the chemical compounds: VSV, immediately, 10000 cells per well; SV40, immediately, 7500 cells per well.

### Screens

Three master plates were prepared using dimethylsulfoxide (DMSO), triethyleneglycol monoethyl ether (TEGME) or a mixture of 30% DMSO and 70% TEGME, depending on compound solubility. The concentration of test compound was 3 mM. The substances were transferred into 96-well glass plates (100 μL; 6×9 format) and were diluted 1:100 prior to addition to the cells.

The screens were divided into cytotoxicity and a functional part, whereby the toxicity profile (comprising Adenylate-kinase release, live/dead assay and apoptosis assay) were performed first in order to assure non-toxic concentrations of substances. According to the results the substances were diluted with the corresponding solvent. The screen was performed in triplicates and repeated two times with the final concentration of the substances for all assays.

The master plates were stored at −20° C. For the preparation of the working solution the library containing plates were defrosted at 37° C. The substances were diluted in D-MEM medium without serum. The medium was removed from the cells and the working solution was added to each of the triplicate plates. Growth control medium was added and additional specific controls for each assay were applied. Finally, serum was supplied to the cells, and the plates were incubated at 37° C. in an atmosphere containing 5% carbon dioxide.

### VSV Infection Assay

VSV-GFP were added immediately after substance addition to the cells in a concentration that gave rise to approximately 50% infected cells. After 4 h incubation the cells were fixed with paraformaldehyde, washed and stained with DRAQ5™. A microscopic analysis with the automated confocal fluorescence microscope OPERA (Evotec Technologies GmbH) was performed, using 488 and 633 nm laser excitation and a water-immersion 20×-objective. In a fully automated manner, 10 images per well were taken, the total number of cells (DRAQ5) and the number of infected cells (VSV-GFP) were determined by automated image analysis and average and standard deviations for triplicates calculated. The VSV infection (in percentage) was calculated by dividing the number of VSV infected nuclei with the total number of nuclei (DRAQ5 stained), multiplied by 100%. The calculated values are expressed as percentage of untreated cells.

### SV40 Infection Assay

Wlld type SV40 viruses were added immediately after substance addition to the cells. After 36 h incubation the cells were fixed with paraformaldehyde, washed and stained with DRAQ5™. A monoclonal antibody directly conjugated to Alexa Fluor 488 was used to detect T-antigen expression. A microscopic analysis with the automated confocal fluorescence microscope OPERA (Evotec Technologies GmbH) was performed, using 488 and 633 nm laser excitation and a water-immersion 20×-objective. In a fully automated manner, 10 images per well were taken, the total number of cells (DRAQ5) and the number of infected cells (monoclonal antibody bound to SV40 T-antigen) were determined by automated image analysis and average and standard deviations for triplicates calculated. The SV40 infection (in percentage) was calculated by dividing the number of SV40 infected nuclei with the total number of nuclei (DRAQ5 stained), multiplied by 100%. The calculated values are expressed as percentage of untreated cells.

### Quantification of Results

The raw data of the SV 40 assay are counts of successfully infected and total cells, determined per well of a 96-well plate. (Total cells are stained by DRAQ5, while the infected cells are counted by specific immuno-histochemical staining of expressed SV-40 T-Antigen as described above). First the ratio of infected to total cells is determined in the following manner.

In each individual assay three wells on three parallel plates per test compound are evaluated, the ratios of infected to total cells are averaged and standard deviation is determined. The data are then transformed to percentages: Controls or solvent controls are set as 100% and data for each test compound are transformed to percentage values with respect to the appropriate solvent control.

Each test compound was subjected to two or three independent assays. The average % controls and % standard deviations are determined as averages of % control and % standard deviations of the individual, independent assays. Finally, the inhibition value is calculated using the following formula:

% inhibition=100−% control

Results: Four of the compounds that tested positive in the biophysical DLRA and thus identified as disrafters, 10ad, 10ac, 10af and 10da, were evaluated for their inhibitory effect in the SV40 infection assay. These compounds provided good results. 10ad inhibited SV40 infection by 15.2% at a concentration of 30 μM, while 10ac inhibited the same process by 12.9% at 30 μM concentration compared to solvent. Similarly, compound 10af inhibited infection by 18.9% (at 30 μM) and compound 10da by 29.6% (at 15 μM). Thus, these substances are preferred compounds for the pharmaceutical intervention in the case of the viral and bacterial infections described above. Another of the compounds which tested positive in the DLRA, i.e. compound 10db, provided for a particular good result in the SV40 assay and is thus a more preferred compound to be used in the pharmaceutical compositions described herein for the treatment of diseases caused by viral or bacterial infections, for whom the SV40 assay may serve as a model for viral or bacterial uptake. Compound 10db inhibited SV40 infection by 52.2% at a concentration of 30 μM compared to solvent vehicle alone. Remarkably, no inhibitory effect on viral infection at all was observed when testing compounds 10ac, 10ad, 10af, 10da and 10db in the VSV counterscreen, thus proving the working hypothesis provided herein for the mode of action of the compounds described in this invention.

### EXAMPLE 19

### HIV Assay

In order to evaluate their specific usefulness for the development of pharmaceutical compositions used for the treatment of Acquired Immune Deficiency Syndrome (AIDS), which is caused by HIV infection, compounds were tested for inhibition of infection of HeLa TZM cells by HIV-1 strain NL4-3 (laboratory adapted B-type strain). TZM is a CD4-positive HIV-infectable HeLa derivative that contains an HIV-1 LTR-driven luciferase reporter gene. HIV-infection leads to production of the viral trans-activator Tat which induces luciferase expression and luciferase activity can thus be used to score for infected cells.

Test compounds were provided as solutions in dimethylsulfoxide (DMSO), triethyleneglycol monoethyl ether (TEGME) or a mixture of 30% DMSO and 70% TEGME, depending on compound solubility. The concentration of test compound in those stock solutions was 3 mM.

All assays were performed in duplicate. Prior to harvest, cells were analyzed by microscopy for visible cytotoxic effects.

In general, infection with HIV-1 NL4-3 led to ca. 5000-10000 arbitrary light units with some variation depending on the experiment and the use of solvent. PBS controls and solvent controls without any virus yielded 100-200 arbitrary light units.

On the first day, around 50000 TZM cells per well were seeded in 48-well plates. Next day compounds were thawed at 37° C., briefly vortexed and diluted 1:100 in cell culture medium directly before addition to tissue culture cells. 2 μL compound solution was added to 148 μL DMEM (containing 10% FCS and antibiotics) and mixed. The medium was removed from TZM cells and 150 μL of compound-containing medium was added. Subsequently, cells were incubated for 24 h at 37° C. in an atmosphere containing 5% carbon dioxide. 50 μL virus (produced from HIV-1, strain NL4-3 infected MT-4 cells) in RPMI1640 medium (containing 10% FCS and antibiotics) were added and cells were incubated for 24 h at 37° C. in an atmosphere containing 5% carbon dioxide. On the third day, the medium was removed, cells were washed once with DMEM, and 100 μL DMEM were added followed by 100 μL Steady-Glo substrate. Cells were incubated for 30-60 min at room temperature, then 180 μL were transferred from the 48-well plate to a 96-well plate, and luciferase activity was measured using a TECAN plate luminometer (5s per well). Both, solvent controls with and without virus were performed.

### Quantification of Results

Each assay plate contains duplicates for each test compound and the appropriate solvent controls. When recording Luminometer readings, a background of uninfected cell controls is subtracted. Duplicates are averaged and converted to % control by dividing the average by the average of the relevant solvent control and multiplying by 100. Assays are repeated once or twice, and final results were determined by averaging the % controls from the two or three independent assays.

Finally, the inhibition value is calculated using the following formula:

% inhibition=100−% control

Results: Three compounds that tested positive in the initial DLRA and thus identified as disrafters, 10ak, 10da and 10db, were evaluated in the HIV infection assay They provided good results. 10ak inhibited HIV infection by 23% at a concentration of 30 μM, while 10da inhibited the same process by 18% at 30 μM concentration compared to solvent. Similarly, compound 10db inhibited infection by 27% (at 30 μM). Thus, these substances are preferred compounds for the pharmaceutical intervention in the case of AIDS. A further compound which tested positive in the DLRA, i.e. compound 10dc, provided for a particular good result in the HIV assay and is thus a more preferred compound to be used in the pharmaceutical compositions described herein for the treatment of AIDS. Compound 10dc inhibited HIV infection in the given experimental setting by 38% at a concentration of 30 μM compared to solvent vehicle alone.

1. A method for the treatment and/or amelioration of a disease/disorder caused by a biochemical/biophysical pathological process occurring on, in or within lipid rafts, the method comprising administering an effective amount of a compound that treats or ameliorates such a disease/disorder to a subject, wherein the compound is a compound of one of the following formulae 1a, 1b, 1c and 1d: 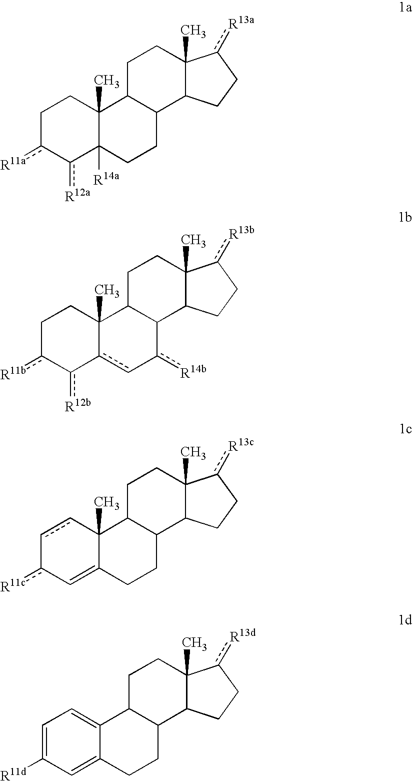
wherein [symbol] is a single bond or a double bond;
R^{11a}, R^{11b} and R^{11c} are H, OR, NR₂, N₃, SO₄⁻, SO₃⁻, PO₄²⁻, halogen, O or S, provided that if R^{11a}, R^{11b} or R^{11c} is O or S then the bond connecting said R^{11a}, R^{11b} or R^{11c} to the ring system is a double bond, in all other cases said bond is a single bond;
R^{11d} is OR, NR₂, SO₄⁻, PO₄²⁻, COOH, CONR₂ or OCO(C₁₋₄ alkyl);
R^{12a} and R^{12b} are H, OR, NR₂, N₃, halogen or O, provided that if R^{12a} or R^{12b} is O then the bond connecting said R^{12a} or R^{12b} to the ring system is a double bond, in all other cases said bond is a single bond;
provided that not both of R^{11a} and R^{12a} are H and provided that not both of R^{11b} and R¹²b are H;
R^{13a}, R^{13b}, R^{13c} and R^{13d} are H; C₁₋₅ alkyl, wherein one or more hydrogens are optionally replaced by halogen; C₁₂₋₂₄ alkyl, wherein one or more hydrogens are optionally replaced by halogen; C₁₋₅ alkylidene, wherein one or more hydrogens are optionally replaced by halogen; C₁₂₋₂₄ alkylidene, wherein one or more hydrogens are optionally replaced by halogen; C₂₋₅ alkenyl, wherein one or more hydrogens are optionally replaced by halogen; C₂₋₅ alkynyl, wherein one or more hydrogens are optionally replaced by halogen; 1-adamantyl; (1-adamantyl)methylene; C₃₋₈ cycloalkyl, wherein one or more hydrogens are optionally replaced by halogen; (C₃₋₈ cycloalkyl)methylene, wherein one or more hydrogens are optionally replaced by halogen; provided that if R^{13a}, R^{13b} or R^{13c} is C₁₋₅ alkylidene or C₁₂₋₂₄ alkylidene then the bond connecting said R^{13a}, R^{13b} or R^{13c} to the ring system is a double bond, in all other above-mentioned cases said bond is a single bond; or
R^{13a}, R^{13b} and R^{13c} are a group of the following formula 2: 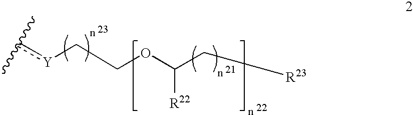
wherein R²³ is O—R²¹ or NH—R²⁴;
R²¹ is C₁₋₄ alkyl, CO(C₁₋₄alkyl) or H;
R²⁴ is C₁₋₄ alkyl, CO(C₁₋₄alkyl) or H;
each R²² is independently H or C₁₋₃ alkyl;
Y is CH₂, CH or O, provided that if Y is CH then the bond connecting Y to the ring system is a double bond, in all other cases said bond is a single bond;
each n²¹ is independently an integer of 1 or 2;
n22 is an integer from 0 to 5;
if Y is O then n²³ is 1, in all other cases n²³ is 0;
R^{14a} is H;
R^{14b} is H, OR, halogen or O, provided that if R^{14b} is O then the bond connecting R^{14b} to the ring system is a double bond, in all other cases said bond is a single bond; and
each R is independently H or C₁₋₄ alkyl;
or a pharmaceutically acceptable salt, or solvate thereof

2. The method of claim 1, wherein R^{11a}, R^{11b} and R^{11c} are OCH₃, NH₂, N(C₁₋₄ alkyl)₂, SO₄⁻or O and wherein R^{11d} is OCH₃, NR₂ or OCOCH₃.

3. The method of claim 1, wherein R^{12a} and R^{12b} are H, O(C₁₋₄ alkyl), halogen or O.

4. The method of claim 1, wherein R ^{13a}, R^{13b}, R^{13c} and R^{13d} are H, C₁₋₅ alkyl, C₁₋₅ alkylidene, C₁₂₋₁₄ alkyl or C₁₂₋₁₄ alkylidene.

5. The method of claim 1, wherein R^{13a}, R^{13b}, R^{13c} and R^{13d} are the group of formula 2.

6. The method of claim 1, wherein R^{14b} is H, halogen or O.

7. The method of claim 1, wherein the compound has one of the following formulae 10aa to 10ae:

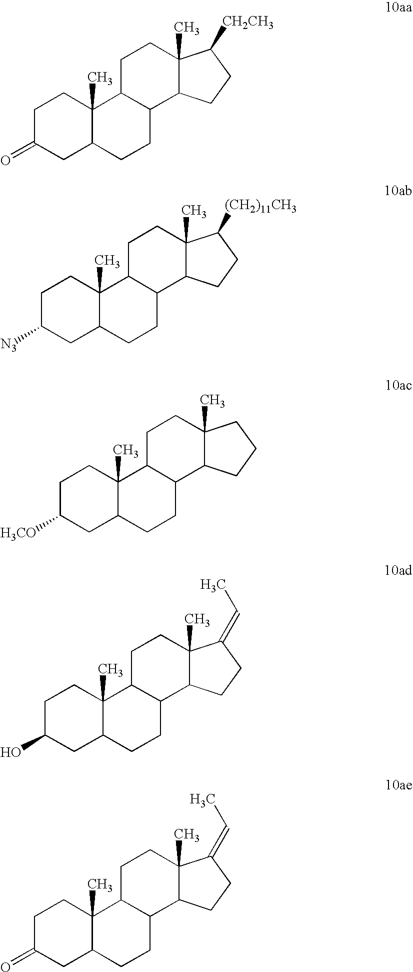

8. The method of claim 1, wherein the compound has one of the following formulae 10af to 10al:

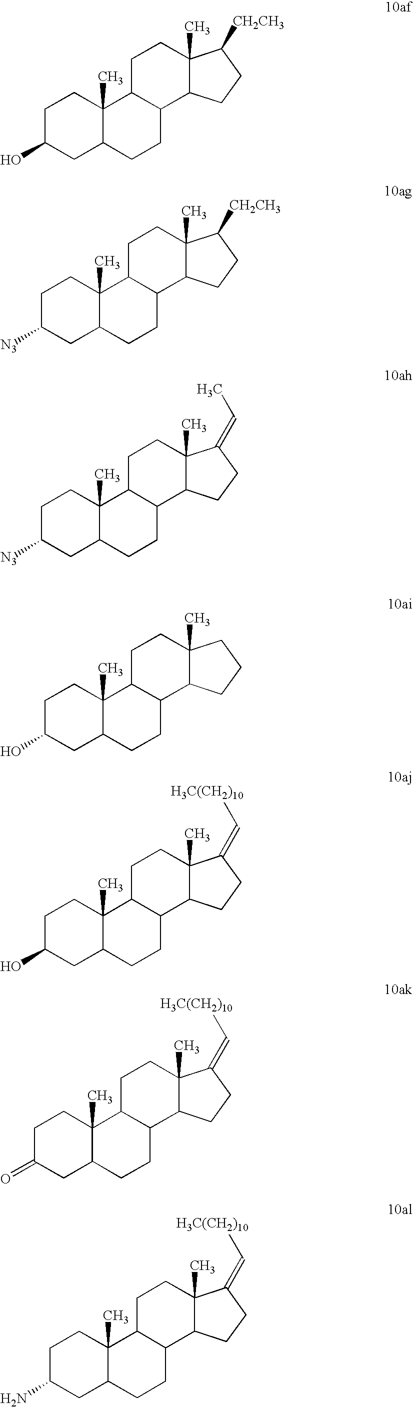

9. The method of claim 1, wherein the compound has one of the following formulae 10da to 10dc:

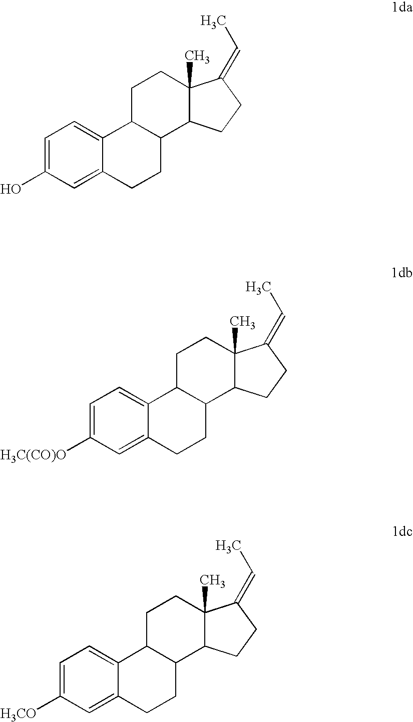

10. The method of claim 1, wherein said disease/disorder caused by a biochemical/biophysical pathological process occurring on, in or within lipid rafts is selected from the group consisting of a neurodegenerative disease, an infectious disease, an immunological disease/disorder, a proliferative disorder and a systemic disease.

11. The method of claim 10, wherein said neurodegenerative disease is Alzheimer's disease or a prion disease.

12. The method of claim 11, wherein said prion disease is selected from the group consisting of Creutzfeldt-Jakob disease, Kuru, Gerstmann-Sträussler-Schneiker syndrome and fatal familial insomnia.

13. The method of claim 10, wherein said infectious disease is caused by a virus, a bacterium or a parasite.

14. The method of claim 13, wherein said virus is selected from the group consisting of influenza, HIV, Hepatitis virus (A, B, C, D), Rotavirus, Respiratory syncytial cell virus, Herpetoviridae (e.g. Herpes simplex virus, Epstein-Barr virus), Echovirus 1, measles virus, Picornaviridae (e.g. Enterovirus, Coxsackie virus), Filoviridae (e.g. Ebolavirus, Marburgvirus), Papillomaviridae and polyomaviridae.

15. The method of claim 13, wherein said bacterium is selected from the group consisting of Mycobacterium tuberculosis, Mycobacterium bovis, Shigella spp., Campylobacter jejuni, Chlamydia pneumoniae, Escherichia coli, Aeromonas hydrophila, Vibrio cholerae, Clostridium difficile, Clostridium tetani, Bacillus anthracis and Heliobacter pylori.

16. The method of claim 13, wherein said parasite is selected from the group consisting of Plasmodium falciparum, Toxoplasma gondii, Trypanosoma and Leishmania.

17. The method of claim 10, wherein said immunological disease/disorder is an autoimmune disease or a hyperallergenic disease.

18. The method of claim 17, wherein the hyperallergenic disease is asthma.

19. The method of claim 17, wherein said autoimmune disease is Batten disease, systemic lupus erythematosus or artheriosclerosis.

20. The method of claim 10, wherein said proliferative disorder is a cancerous disease.

21. The method of claim 10, wherein said systemic disease is diabetes.

22. The method of claim 14, wherein the compound has formula 10ad, 10ae, 10af or 10al and the method comprises the and/or amelioration of an influenza infection.

23. The method of claim 14, wherein the compound has formula 10ak, 10da, 10db or 10dc and the method comprises the and/or amelioration of an HIV infection.

24. The method of claim 18, wherein the compound has formula 10al and the method comprises the treatment, and/or amelioration of asthma

25. A pharmaceutical composition comprising as an active ingredient a compound of one of the following formulae 1a, 1b, 1c and 1d: 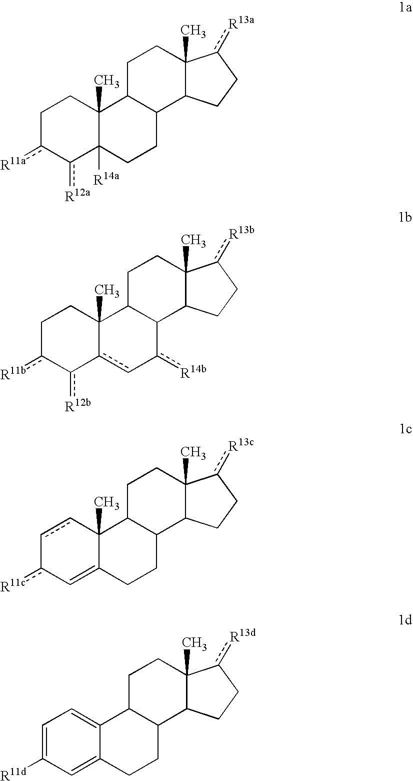
wherein [symbol]
is a single bond or a double bond;
R^{11a}, R^{11b} and R¹¹c are H, OR, NR₂, N₃, SO₄⁻, SO₃⁻, PO₄²⁻, halogen, O or S, provided that if R^{11a}, R^{11b} or R^{11c} is O or S then the bond connecting said R^{11a}, R^{11b} or R^{11c} to the ring system is a double bond, in all other cases said bond is a single bond;
R^{11d} is OR, NR₂, SO₄⁻, PO₄²⁻, COOH, CONR₂ or OCO(C₁₋₄ alkyl);
R^{12a} and R^{12b} are H, OR, NR₂, N₃, halogen or O, provided that if R^{12a} or R^{12b} is O then the bond connecting said R^{12a} or R^{12b} to the ring system is a double bond, in all other cases said bond is a single bond;
provided that not both of R^{11a} and R^{12a} are H and provided that not both of R^{11b} and R^{12b} are H;
R^{13a}, R^{13b}, R^{13c} and R^{13d} are H; C₁₋₅ alkyl, wherein one or more hydrogens are optionally replaced by halogen; C₁₂₋₂₄ alkyl, wherein one or more hydrogens are optionally replaced by halogen; C₁₋₅ alkylidene, wherein one or more hydrogens are optionally replaced by halogen; C₁₂₋₂₄ alkylidene, wherein one or more hydrogens are optionally replaced by halogen; C₂₋₅ alkenyl, wherein one or more hydrogens are optionally replaced by halogen; C₂₋₅ alkynyl, wherein one or more hydrogens are optionally replaced by halogen; 1-adamantyl; (1-adamantyl)methylene; C₃₋₈ cycloalkyl, wherein one or more hydrogens are optionally replaced by halogen; (C₃₋₈ cycloalkyl)methylene, wherein one or more hydrogens are optionally replaced by halogen; provided that if R^{13a}, R^{13b} or R^{13c} is C₁₋₅ alkylidene or C₁₂₋₂₄ alkylidene then the bond connecting said R^{13a}, R^{13b} or R^{13c} to the ring system is a double bond, in all other above-mentioned cases said bond is a single bond; or
R^{13a}, R^{13b} and R^{13c} are a group of the following formula 2: 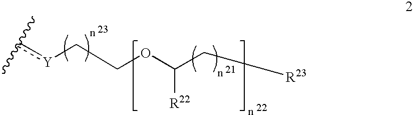
wherein R²³ is O—R²¹ or NH—R²⁴;
R²¹ is C₁₋₄ alkyl, CO(C₁₋₄alkyl) or H;
R²⁴ is C₁₋₄ alkyl, CO(C₁₋₄alkyl) or H;
each R²² is independently H or C₁₋₃ alkyl;
Y is CH₂, CH or O, provided that if Y is CH then the bond connecting Y to the ring system is a double bond, in all other cases said bond is a single bond;
each n²¹ is independently an integer of 1 or 2;
n22 is an integer from 0 to 5;
if Y is O then n²³ is 1, in all other cases n²³ is O;
R^{14a} is H;
R^{14b} is H, OR, halogen or O, provided that if R^{14b} is O then the bond connecting R^{14b}to the ring system is a double bond, in all other cases said bond is a single bond; and
each R is independently H or C₁₋₄ alkyl;
or a pharmaceutically acceptable salt, or solvate thereof.
